(19) United States
(12) Patent Application Publication
(10) Pub. No.: US 2014/0230081 A1
(43) Pub. Date: Aug. 14, 2014

(54) POLYNUCLEOTIDE, POLYPEPTIDE, ANIMAL MODEL AND METHOD FOR THE DEVELOPMENT OF COMPLEMENT SYSTEM MODULATORS

(71) Applicants: Stefan Golz, Mulheim an der Ruhr (DE); Andreas Geerts, Wuppertal (DE); Maria Köllnberger, Velbert (DE)

(72) Inventors: Stefan Golz, Mulheim an der Ruhr (DE); Andreas Geerts, Wuppertal (DE); Maria Köllnberger, Velbert (DE)

(73) Assignee: BAYER INTELLECTUAL PROPERTY GMBH, Monheim (DE)

(21) Appl. No.: 14/237,880

(22) PCT Filed: Aug. 6, 2012
(86) PCT No.: PCT/EP2012/065359

§ 371 Date: Apr. 10, 2014

(30) Foreign Application Priority Data
Aug. 8, 2011 (EP) 11176800.8

(51) Int. Cl. C07K 14/705; G01N 33/50; G01N 33/566

(52) U.S. Cl. CPC C07K 14/705; G01N 33/566; G01N 33/5008; G01N 2500/10
USPC 800/9; 536/23.5; 530/350; 435/7.21; 436/86; 435/29

(57) ABSTRACT

The present invention is in the field of molecular biology, more particularly, the present invention relates to nucleic acid sequences and amino acid sequences of a hamster C5aR1 and the use of hamster as an animal model for the characterization of complement system modulators within drug discovery.

### TECHNICAL FIELD OF THE INVENTION

The present invention is in the field of molecular biology, more particularly, the present invention relates to nucleic acid sequences and amino acid sequences of a hamster C5aR1 and the use of hamster as an animal model for the characterization of complement system modulators within drug discovery.

### BACKGROUND OF THE INVENTION

### The Complement System

The complement system is a biochemical cascade that helps, or “complements”, the ability of antibodies to clear pathogens from an organism. It is part of the immune system called the innate immune system that is not adaptable and does not change over the course of an individual's lifetime. However, it can be recruited and brought into action by the adaptive immune system.

The complement system consists of a number of small proteins found in the blood, generally synthesized by the liver, and normally circulating as inactive precursors (pro-proteins). When stimulated by one of several triggers, proteases in the system cleave specific proteins to release cytokines and initiate an amplifying cascade of further cleavages. The end-result of this activation cascade is massive amplification of the response and activation of the cell-killing membrane attack complex. Over 25 proteins and protein fragments make up the complement system, including serum proteins, serosal proteins, and cell membrane receptors. These proteins are synthesized mainly in the liver, and they account for about 5% of the globulin fraction of blood serum. Some of the important proteins of the complement are C5 and its products C5a and C5b.

Three biochemical pathways activate the complement system: the classical complement pathway, the alternative complement pathway, and the mannose-binding lectin pathway [1]. The proteins and glycoproteins that constitute the complement system are synthesized by the liver hepatocytes. But significant amounts are also produced by tissue macrophages, blood monocytes and epithelial cells of the genitourinal tract and gastrointestinal tract. The three pathways all generate homologous variants of the protease C3-convertase. The classical complement pathway typically requires antibodies for activation (specific immune response), whereas the alternative and mannose-binding lectin pathways can be activated by C3 hydrolysis or antigens without the presence of antibodies (non-specific immune response). In all three pathways, a C3-convertase cleaves and activates component C3, creating C3a and C3b and causing a cascade of further cleavage and activation events. C3b binds to the surface of pathogens leading to greater internalization by phagocytic cells by opsonization. C5a is an important chemotactic protein, helping recruit inflammatory cells. Both C3a and C5a have anaphylatoxin activity, directly triggering degranulation of mast cells as well as increasing vascular permeability and smooth muscle contraction. C5b initiates the membrane attack pathway, which results in the membrane attack complex (MAC), consisting of C5b, C6, C7, C8, and polymeric C9 [2]. MAC is the cytolytic endproduct of the complement cascade; it forms a transmembrane channel, which causes osmotic lysis of the target cell. Kupffer cells and other macrophage cell types help clear complement-coated pathogens. As part of the innate immune system, elements of the complement cascade can be found in species earlier than vertebrates; most recently in the protostome horseshoe crab species, putting the origins of the system back further than was previously thought.

The classical pathway is triggered by activation of the C1-complex (C1q, two molecules of C1r, and two molecules of C1s thus forming C1qr2s2), which occurs when C1q binds to IgM or IgG complexed with antigens (a single IgM can initiate the pathway, while multiple IgGs are needed), or when C1q binds directly to the surface of the pathogen. Such binding leads to conformational changes in the C1q molecule, which leads to the activation of two C1r (a serine protease) molecules. They then cleave C1s (another serine protease). The C1r2s2 component now splits C4 and then C2, producing C4a, C4b, C2a, and C2b. C4b and C2a bind to form the classical pathway C3-convertase (C4b2a complex), which promotes cleavage of C3 into C3a and C3b; C3b later joins with C4b2a (the C3 convertase) to make C5 convertase (C4b2a3b complex). The inhibition of C1r and C1s is controlled by C1-inhibitor. C3-convertase can be inhibited by Decay accelerating factor (DAF), which is bound to erythrocyte plasma membranes via a GPI anchor.

The alternative pathway is triggered by spontaneous C3 hydrolysis directly due to the breakdown of the thioester bond via condensation reaction (C3 is mildly unstable in aqueous environment) to form C3a and C3b. It does not rely on a pathogen-binding antibodies like the other s. [1]. C3b is then capable of covalently binding to a pathogenic membrane surface if it is near enough. If there is no pathogen in the blood, the C3a and C3b protein fragments will be deactivated by rejoining with each other. Upon binding with a cellular membrane C3b is bound by factor B to form C3bB. This complex in presence of factor D will be cleaved into Ba and Bb. Bb will remain covalently bonded to C3b to form C3bBb which is the alternative pathway C3-convertase. The protein C3 is produced in the liver. The C3bBb complex, which is “hooked” onto the surface of the pathogen, will then act like a “chain saw,” catalyzing the hydrolysis of C3 in the blood into C3a and C3b, which positively affects the number of C3bBb hooked onto a pathogen. After hydrolysis of C3, C3b complexes to become C3bBbC3b, which cleaves C5 into C5a and C5b. C5b with C6, C7, C8, and C9 (C5b6789) complex to form the membrane attack complex, also known as MAC, which is inserted into the cell membrane, “punches a hole,” and initiates cells lysis. C5a and C3a are known to trigger mast cell degranulation.

The lectin pathway is homologous to the classical pathway, but with the opsonin, mannose-binding lectin (MBL), and ficolins, instead of C1q. This pathway is activated by binding mannose-binding lectin to mannose residues on the pathogen surface, which activates the MBL-associated serine proteases, MASP-1, and MASP-2 (very similar to C1r and C1s, respectively), which can then split C4 into C4a and C4b and C2 into C2a and C2b. C4b and C2a then bind together to form the C3-convertase, as in the classical pathway. Ficolins are homologous to MBL and function via MASP in a similar way. In invertebrates without an adaptive immune system, ficolins are expanded and their binding specificities diversified to compensate for the lack of pathogen-specific recognition molecules.

### Role in Disease

It is thought that the complement system might play a role in many diseases with an immune component, such as Barraquer-Simons Syndrome, asthma, lupus erythematosus, glomerulonephritis, various forms of arthritis, autoimmune heart disease, multiple sclerosis, inflammatory bowel disease, and ischemia-reperfusion injuries. The complement system is also becoming increasingly implicated in diseases of the central nervous system such as Alzheimer's disease and other neurodegenerative conditions.

Deficiencies of the terminal pathway predispose to both autoimmune disease and infections (particularly Neisseria meningitis, due to the role that the C5 complex plays in attacking Gram-negative bacteria). Mutations in the complement regulators factor H and membrane cofactor protein have been associated with atypical haemolytic uraemic syndrome. Moreover, a common single nucleotide polymorphism in factor H (Y402H) has been associated with the common eye disease age-related macular degeneration. Both of these disorders are currently thought to be due to aberrant complement activation on the surface of host cells. Mutations in the C1 inhibitor gene can cause hereditary angioedema, an autoimmune condition resulting from reduced regulation of the complement pathway.

### Complement Component 5a (C5a)

C5a is a protein fragment released from complement component C5. In humans, the polypeptide contains 74 amino acids. NMR spectroscopy proved that the molecule is composed of four helices and loops connecting the helices. On the N terminus a short 1.5 turn helix is also present [3]. The longest helix—IV—develops three disulfide bonds with helix II and III. C5a is rapidly metabolised by a serum enzyme, carboxypeptidase B to a 73 amino acid form, C5a-des-Arg.

Complement Component 5a Receptor (C5aR1)

The split product of the complement protein, C5, is C5a and is an extremely potent pro-inflammatory peptide that interacts with two C5a receptors, C5aR and C5L2, present on surfaces of phagocytes as well as other cell types. The former is a well-established receptor that initiates G-protein-coupled signaling via mitogen-activated protein kinase pathways. Its in vivo blockade greatly reduces inflammatory injury. Much less is known about C5L2, occupancy of which by C5a does not initiate increased intracellular Ca2+. There are numerous conflicting reports suggesting that C5L2 is a “default receptor” that attenuates C5a-dependent biological responses by competing with CSaR for binding of C5a. However, there are other reports suggesting that C5L2 plays an active, positive role in inflammatory responses. Better definition of C5L2 is needed if its in vivo blockade, along with CSaR, is to be considered in complement-dependent diseases [5]. The initial structural characterization in 1991 [6, 7] of the rhodopsin-like receptor for C5a, C5aR [6], and the receptor for N-formyl Met-Leu-Phe [7] provided key biochemical information that would permit development of antibodies and synthetic inhibitors to these receptors, for which C5aR binds C5a with high affinity and initiates G-protein dependent cascade of cell responses (increased intracellular Ca2+, granule fusion with the cell membrane, enzyme release, on oxidative burst [H2O2 production], etc.). Similar signaling events occur with receptor-ligand interaction involving the formyl peptide receptor. CSaR is now known to be crucial in the initiation of acute inflammatory responses [8, 9]. In the early 2000s, a second receptor for C5a, C5L2, was described, based on its ability to bind C5a and C5a-des-Arg with high affinity in the absence of an intracellular Ca2+ signal [10]. However, signaling as assessed by phosphoprotein appearance in myeloid-derived cells (neutrophils [PMNs], macrophages, monocytes, and dendritic cells) could not be measured. Functional responses, such as chemotaxis, enzyme release, the respiratory burst, etc., were also undetectable after ligation of C5L2 with C5a, leading to the designation of C5L2 as a “default” or “scavenger” receptor [11]. In other words, C5L2 competed with C5aR for binding of C5a, and the balance in C5a occupancy between the two receptors would determine the outcome (pro-inflammatory or anti-inflammatory).

C5aR1 is described to be involved, but not limited to, in the following diseases, disorders and processes:

Alzheimer [12], neurodegenerative disease [13], sepsis [13, 16], adaptive immune responses [13], allergic asthma [13], transplantation [13], cancer [13], T-cell activation [13], autoimmune diseases [13], inflammatory bowel disease [14], organ protection [15], Acute Respiratory Distress Syndrome (ARDS) [17], anti-complement therapy [18], paroxysmal nocturnal hemoglobinuria [18], Glomerulonephritis [18], Cardiac surgery, acute myocardial infarction treated with thrombolysis [18], acute myocardial infarction treated with angioplasty [18], acute myocardial infarction treated with cardiopulmonary bypass [18], stable coronary artery disease [18], Coronary artery bypass graft surgery [18], ischemia-reperfusion injury [18], cardiopulmonary bypass [18], age-related macular degeneration [18], heart failure [19], abdominal aortic aneurysm [20], acute renal failure [21]

### Drug Discovery

During the process of drug design, medicinal chemists need to solve three basic problems: lead compound identification; lead optimization elevating the lead into candidate drug status; and, following detailed pharmacological studies, the improvement of pharmacokinetic and pharmacodynamic properties of the future drug. Traditionally, natural products, synthetic compounds, human metabolites, metabolites of drugs, known drugs, analogs of the transition state of enzymatic reactions and suicidal inhibitors of enzymes are used as sources of lead structures. In the past few decades, powerful experimental methods have sped up the search for lead structures. HTS (simultaneous testing in vitro of hundreds and thousands of compounds from libraries of chemical structures) is used for identification of ‘hits’, molecules that strongly bind the selected enzymes or receptors. To become leads these compounds need to have lead-like properties and, subsequently, to confirm their activity in more elaborate biological assays. Another experimental approach makes use of combinatorial chemistry, where tens and hundreds of compounds from building blocks are synthesized in parallel and then tested for activity, using automated systems. Recently, the dynamic combinatorial chemistry has developed quickly, which implies addition of the target enzyme or receptor to the reactive system, thus creating a driving force that favors the formation of the best binding combination of building blocks. This selfscreening process accelerates the identification of lead compounds for drug discovery. If the 3D structure of the biological target is available from X-ray crystallography and the active site is known, methods of structure-based drug design (SBDD) can be applied for lead identification. There are two basic strategies for searching for biologically active compounds by SBDD: molecular database screening and de novo ligand design. During screening, the different compounds from databases are docked to the active site of a target. The docking program generates hypotheses of probable spatial space, is widely used. Analysis of 3D-QSAR models is carried out by using contour maps of different fields, showing favorable and unfavorable regions for ligand interaction. The QSAR modeling methods allow estimating probable pharmacological activity of unknown compounds. The ‘classical’ QSAR is effective for the development of analogues close to the compounds under modeling. The 3D-QSAR methods are capable of predicting the pharmacological activity of compounds from different chemical classes. Converting a drug candidate with good in vitro properties into a drug with sufficient in vivo properties (for example, decrease in toxicity, increase in solubility, chemical stability and biological half-life) is the third stage of the drug design process. The approaches used in this stage include: the introduction of bioisosters; the design of prodrugs transforming themselves into an active form in the body; twin drugs carrying two pharmacophore groups that bind to one molecule; and soft drugs, which have a pharmacological action localized in specific organs (their distribution in other organs gives rise to metabolic destruction or inactivation) [4].

To date, C5aR has been cloned from human, rat, mouse, dog, rabbit, guinea pig, pig, sheep and several non-human primates (partial). Interestingly, C5aR sequence homology across these various species is unusually divergent. Overall C5aR sequence homology is 95% between human and non-human primate. Conversely, between human and non-primate C5aR5, homology is only 65-75%. These differences are unusual for G-protein-coupled receptors, which are typically 85-95% homologous across species. All full-length, recombinant and natively expressed C5aR5, except rat, bind human C5a with high affinity, suggesting relative conservation of C5a ligand-binding domains. However, cyclic peptide and small molecule C5aR antagonists demonstrate a greater degree of species selectivity. This suggests different C5aR binding and activation determinants for C5a peptide and small molecule antagonists. A small molecule C5aR antagonist (W-54011; CAS number: 405098-33-1) inhibits C5a-mediated responses in human, cynomolgus monkey, and gerbil neutrophils, but not in mouse, rat, guinea pig, rabbit, or dog neutrophils.

Because of this observed small molecule antagonist species selectivity could be responsible for the observed species-selective pharmacology [23].

The pharmaceutical industry and biotechnology companies are now heavily focussed on using tools that can provide a better understanding of the function or product of a gene, and that enable the rapid identification and validation of a human drug target among numerous potential candidates. Potential therapeutics could be not only small chemical drug molecules that modulate the function of a protein but also the gene products themselves. The use of phylogenetically lower model organisms to mimic human diseases has become very popular as it enables either the identification of a human gene product (or pathway) that is directly involved in a disease state, or the development of biological screens for molecules or gene products that suppress the disease or stop its progression. The mouse, despite its very low throughput, remains the organism of choice for many close functional parallels with human diseases [24]. For complement related diseases and processes it is necessary to use specific animal models with “human-like” pharmacology due to the known species-selective pharmacology.

Due to the known species-selective pharmacology, the identification of animal species which could be used as animal models is necessary for the development of C5a addressing drugs. The known species with a human-like C5aR amino acid sequence can only be used as animal models with considerable limitations (e.g., but not limited to: housing of the animal, unknown or partially known genomic sequence, limited knowledge about pathophysiology, costs (animals), availability (animals)). Therefore, there is a high need for the identification of an animal model suitable for C5a directed pharmacological research and development.

### SUMMARY OF THE INVENTION

The invention relates to the use of hamster as an animal model for the characterization of complement system modulators within drug discovery. The invention relates to the use of hamster as an animal model for the characterization of C5aR1 modulators within drug discovery.

The invention relates to the use of hamster as an animal model for the characterization of C5a modulators within drug discovery.

The invention relates to the use of hamster as an animal model for the characterization of C5b modulators within drug discovery.

The invention relates to the use of hamster as an animal model for the characterization of fragments of C5 modulators within drug discovery.

The invention relates to the use of hamster as an animal model for the characterization of polypeptides of C5 modulators within drug discovery.

The invention relates to the use of hamster as an animal model for the characterization of C5 modulators within drug discovery.

The invention relates to the use of hamster as an animal model for the characterization of C3aR1 modulators within drug discovery.

The invention relates to the use of hamster as an animal model for the characterization of C3a modulators within drug discovery.

The invention relates to the nucleotide sequence of hamster C5aR1.

The invention relates to the polypeptide sequence of hamster C5aR1.

The invention relates to the use of recombinant expressed hamster C5aR1.

The invention relates to the use of hamster C5aR1 in in vitro assays, as, but not limited to binding assays, activity assays, cell based assays and cell-free assays.

The invention relates to the use of hamster C5a in in vitro assays, as, but not limited to binding assays, activity assays, cell based assays and cell-free assays.

The invention relates to the use of hamster C5b in in vitro assays, as, but not limited to binding assays, activity assays, cell based assays and cell-free assays.

The invention relates to the use of hamster C5 in in vitro assays, as, but not limited to binding assays, activity assays, cell based assays and cell-free assays.

The invention relates to the use of hamster C5aR1 in assays to characterize or analyze the interaction of C5aR1 with it ligands or agonists.

The invention relates to the use of hamster C5aR1 in assays to characterize compound, peptides or antibodies which modify the activity of C5aR1.

### BRIEF DESCRIPTION OF THE DRAWINGS

FIG. 1 shows the nucleotide sequence of hamster C5aR1 polynucleotide (SEQ ID NO:1).

FIG. 2 shows the amino acid sequence of hamster C5aR1 polypeptide (SEQ ID NO:2).

FIG. 3 shows the nucleotide sequence of primer human C5aR1 (SEQ ID NO:3).

FIG. 4 shows the nucleotide sequence of primer human C5aR1 (SEQ ID NO4).

FIG. 5 shows the nucleotide sequence of primer human C5aR1 (SEQ ID NO:5).

FIG. 6 shows expression of IL10 in kidney of CLP model in hamster (X axis=control: no CLP, no treatment; sham+vehicle: sham abdominal surgery+treatment with vehicle; CLP+vehicle: CLP+treatment with vehicle; CLP+0.5 mg/kg: CLP+treatment with 0.5 mg/kg W54011; CLP+2.5 mg/kg: CLP+treatment with 2.5 mg/kg W54011; CLP+Alzet: CLP+treatment with W54011/administration by alzet pump; Y-axis=relative expression)

FIG. 7 shows expression of IL10 in LV (left ventricle) of CLP model in hamster (X axis=control: no CLP, no treatment; sham+vehicle: sham abdominal surgery+treatment with vehicle; CLP+vehicle: CLP+treatment with vehicle; CLP+0.5 mg/kg: CLP+treatment with 0.5 mg/kg W54011; CLP+2.5 mg/kg: CLP+treatment with 2.5 mg/kg W54011; CLP+Alzet: CLP+treatment with W54011/administration by alzet pump; Y-axis=relative expression)

FIG. 8 shows expression of IL10 in lung of CLP model in hamster (X axis=control: no CLP, no treatment; sham+vehicle: sham abdominal surgery+treatment with vehicle; CLP+vehicle: CLP+treatment with vehicle; CLP+0.5 mg/kg: CLP+treatment with 0.5 mg/kg W54011; CLP+2.5 mg/kg: CLP+treatment with 2.5 mg/kg W54011; CLP+Alzet: CLP+treatment with W54011/administration by alzet pump; Y-axis=relative expression)

FIG. 9 shows expression of IL6 in LV (left ventricle) of CLP model in hamster (X axis=control: no CLP, no treatment; sham+vehicle: sham abdominal surgery+treatment with vehicle; CLP+vehicle: CLP+treatment with vehicle; CLP+0.5 mg/kg: CLP+treatment with 0.5 mg/kg W54011; CLP+2.5 mg/kg: CLP+treatment with 2.5 mg/kg W54011; CLP+Alzet: CLP+treatment with W54011/administration by alzet pump; Y-axis=relative expression)

FIG. 10 shows expression of IL6 in lung of CLP model in hamster (X axis=control: no CLP, no treatment; sham+vehicle: sham abdominal surgery+treatment with vehicle; CLP+vehicle: CLP+treatment with vehicle; CLP+0.5 mg/kg: CLP+treatment with 0.5 mg/kg W54011; CLP+2.5 mg/kg: CLP+treatment with 2.5 mg/kg W54011; CLP+Alzet: CLP+treatment with W54011/administration by alzet pump; Y-axis=relative expression)

FIG. 11 shows expression of IL1b in LV (left ventricle) of CLP model in hamster (X axis=control: no CLP, no treatment; sham+vehicle: sham abdominal surgery+treatment with vehicle; CLP+vehicle: CLP+treatment with vehicle; CLP+0.5 mg/kg: CLP+treatment with 0.5 mg/kg W54011; CLP+2.5 mg/kg: CLP+treatment with 2.5 mg/kg W54011; CLP+Alzet: CLP+treatment with W54011/administration by alzet pump; Y-axis=relative expression)

FIG. 12 shows nucleotide sequence of SEQ ID NO:6 for hamster IL10

FIG. 13 shows nucleotide sequence of SEQ ID NO:7 for hamster IL10

FIG. 14 shows nucleotide sequence of SEQ ID NO:8 for hamster IL10

FIG. 15 shows nucleotide sequence of SEQ ID NO:9 for hamster L32

FIG. 16 shows nucleotide sequence of SEQ ID NO:10 for hamster L32

FIG. 17 shows nucleotide sequence of SEQ ID NO:11 for hamster L32

FIG. 18 shows nucleotide sequence of SEQ ID NO:12 for hamster IL1b

FIG. 19 shows nucleotide sequence of SEQ ID NO:13 for hamster IL1b

FIG. 20 shows nucleotide sequence of SEQ ID NO:14 for hamster IL1b

FIG. 21 shows nucleotide sequence of SEQ ID NO:15 for hamster IL6

FIG. 22 shows nucleotide sequence of SEQ ID NO:16 for hamster IL6

FIG. 23 shows nucleotide sequence of SEQ ID NO:17 for hamster IL6

FIG. 24 shows white blood cell counts of EDTA whole blood in hamster CLP model (X axis=control: no CLP, no treatment; sham+vehicle: sham abdominal surgery+treatment with vehicle; CLP+vehicle: CLP+treatment with vehicle; CLP+0.5 mg/kg: CLP+treatment with 0.5 mg/kg W54011; CLP+2.5 mg/kg: CLP+treatment with 2.5 mg/kg W54011; CLP+Alzet: CLP+treatment with W54011/administration by alzet pump; Y-axis=white blood cell counts in % (WBC [%])

FIG. 25 shows the alignment the amino acids sequences of human C5aR1 with hamster C5aR1. The amino acids are given in the one letter code (A: alanine, C: cysteine, D: aspartic acid, E: glutamic acid, F: phenylalanine, G: glycine: H: histidine, I: isoleucine, K: lysine, L: leucine, M: methionine, N: asparagine, P: proline, Q: glutamine, R: arginine, S: serine, T: threonine, V: valine, W: tryptophan, Y: tyrosine. The amino acid position is given by numbers.

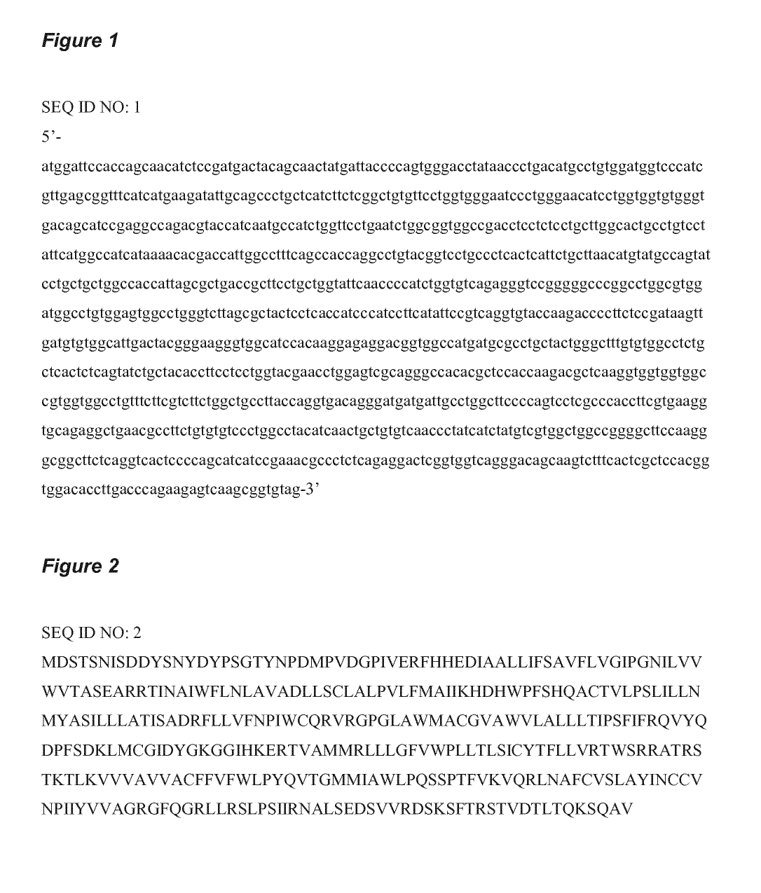

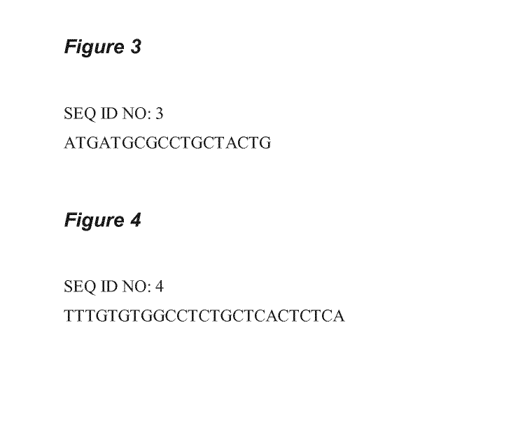

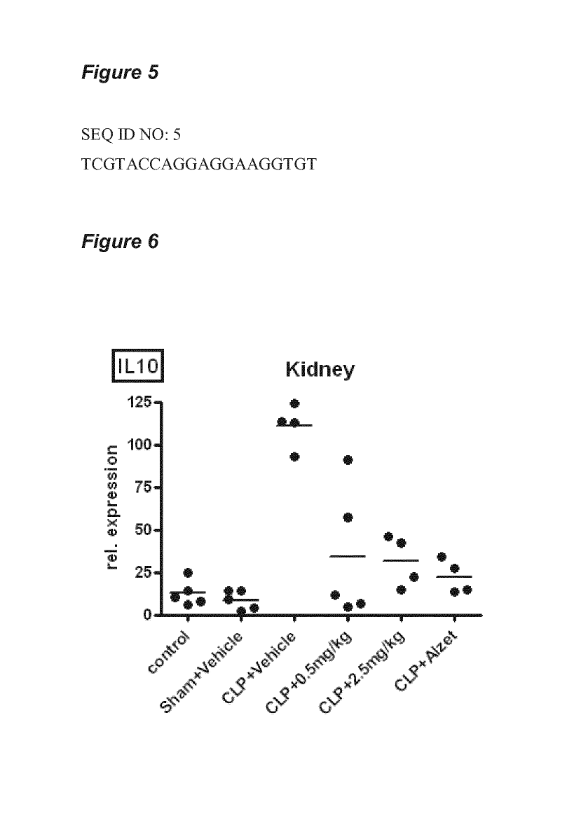

### DETAILED DESCRIPTION OF THE INVENTION

### Definitions of Terms

An “oligonucleotide” is a stretch of nucleotide residues which has a sufficient number of bases to be used as an oligomer, amplimer or probe in a polymerase chain reaction (PCR). Oligonucleotides are prepared from genomic or cDNA sequence and are used to amplify, reveal, or confirm the presence of a similar DNA or RNA in a particular cell or tissue. Oligonucleotides or oligomers comprise portions of a DNA sequence having at least about 10 nucleotides and as many as about 35 nucleotides, preferably about 25 nucleotides.

“Probes” may be derived from naturally occurring or recombinant single- or double-stranded nucleic acids or may be chemically synthesized. They are useful in detecting the presence of identical or similar sequences. Such probes may be labeled with reporter molecules using nick translation, Klenow fill-in reaction, PCR or other methods well known in the art. Nucleic acid probes may be used in southern, northern or in situ hybridizations to determine whether DNA or RNA encoding a certain protein is present in a cell type, tissue, or organ.

A “fragment of a polynucleotide” is a nucleic acid that comprises all or any part of a given nucleotide molecule, the fragment having fewer nucleotides than about 6 kb, preferably fewer than about 1 kb.

“Reporter molecules” are radionuclides, enzymes, fluorescent, chemiluminescent, or chromogenic agents which associate with a particular nucleotide or amino acid sequence, thereby establishing the presence of a certain sequence, or allowing for the quantification of a certain sequence.

“Chimeric” molecules may be constructed by introducing all or part of the nucleotide sequence of this invention into a vector containing additional nucleic acid sequence which might be expected to change any one or several of the following C5AR1 characteristics: cellular location, distribution, ligand-binding affinities, interchain affinities, degradation/turnover rate, signaling, etc.

“Active”, with respect to a C5AR1 polypeptide, refers to those forms, fragments, or domains of a C5AR1 polypeptide which retain the biological activity of a C5AR1 polypeptide, i.e. the biological response to the C5a ligand (e.g. measured by a functional assay).

“Naturally occurring C5AR1 polypeptide” refers to a polypeptide produced by cells which have not been genetically engineered and specifically contemplates various polypeptides arising from post-translational modifications of the polypeptide including but not limited to acetylation, carboxylation, glycosylation, phosphorylation, lipidation and acylation.

“Derivative” refers to polypeptides which have been chemically modified by techniques such as ubiquitination, labeling (see above), pegylation (derivatization with polyethylene glycol), and chemical insertion or substitution of amino acids such as ornithine which do not normally occur in human proteins.

“Conservative amino acid substitutions” result from replacing one amino acid with another having similar structural and/or chemical properties, such as the replacement of a leucine with an isoleucine or valine, an aspartate with a glutamate, or a threonine with a serine.

“Insertions” or “deletions” are typically in the range of about 1 to 5 amino acids. The variation allowed may be experimentally determined by producing the peptide synthetically while systematically making insertions, deletions, or substitutions of nucleotides in the sequence using recombinant DNA techniques.

A “signal sequence” or “leader sequence” can be used, when desired, to direct the polypeptide through a membrane of a cell. Such a sequence may be naturally present on the polypeptides of the present invention or provided from heterologous sources by recombinant DNA techniques.

An “oligopeptide” is a short stretch of amino acid residues and may be expressed from an oligonucleotide. Oligopeptides comprise a stretch of amino acid residues of at least 3, 5, 10 amino acids and at most 10, 15, 25 amino acids, typically of at least 9 to 13 amino acids, and of sufficient length to display biological and/or antigenic activity.

“Inhibitor” is any substance which retards or prevents a chemical or physiological reaction or response. Common inhibitors include but are not limited to antisense molecules, antibodies, and antagonists.

“Biomarker” are measurable and quantifiable biological parameters (e.g. specific enzyme concentration, specific hormone concentration, specific gene phenotype distribution in a population, presence of biological substances) which serve as indices for health—and physiology related assessments, such as disease risk, psychiatric disorders, environmental exposure and its effects, disease diagnosis, metabolic processes, substance abuse, pregnancy, cell line development, epidemiologic studies, etc. Parameter that can be used to identify a toxic effect in an individual organism and can be used in extrapolation between species. Indicator signalling an event or condition in a biological system or sample and giving a measure of exposure, effect, or susceptibility.

Biological markers can reflect a variety of disease characteristics, including the level of exposure to an environmental or genetic trigger, an element of the disease process itself, an intermediate stage between exposure and disease onset, or an independent factor associated with the disease state but not causative of pathogenesis. Depending on the specific characteristic, biomarkers can be used to identify the risk of developing an illness (antecedent biomarkers), aid in identifying disease (diagnostic biomarkers), or predict future disease course, including response to therapy (prognostic biomarkers).

“Standard expression” is a quantitative or qualitative measurement for comparison. It is based on a statistically appropriate number of normal samples and is created to use as a basis of comparison when performing diagnostic assays, running clinical trials, or following patient treatment profiles.

“Animal” as used herein may be defined to include human, domestic (e.g., cats, dogs, etc.), agricultural (e.g., cows, horses, sheep, etc.) or test species (e.g., mouse, rat, rabbit, etc.).

The nucleotide sequences encoding a C5aR1 (or their complement) have numerous applications in techniques known to those skilled in the art of molecular biology. These techniques include use as hybridization probes, use in the construction of oligomers for PCR, use for chromosome and gene mapping, use in the recombinant production of C5aR1, and use in generation of antisense DNA or RNA, their chemical analogs and the like. Uses of nucleotides encoding a C5aR1 disclosed herein are exemplary of known techniques and are not intended to limit their use in any technique known to a person of ordinary skill in the art. Furthermore, the nucleotide sequences disclosed herein may be used in molecular biology techniques that have not yet been developed, provided the new techniques rely on properties of nucleotide sequences that are currently known, e.g., the triplet genetic code, specific base pair interactions, etc.

It will be appreciated by those skilled in the art that as a result of the degeneracy of the genetic code, a multitude of C5aR1-encoding nucleotide sequences may be produced. Some of these will only bear minimal homology to the nucleotide sequence of the known and naturally occurring C5R1. The invention has specifically contemplated each and every possible variation of nucleotide sequence that could be made by selecting combinations based on possible codon choices. These combinations are made in accordance with the standard triplet genetic code as applied to the nucleotide sequence of naturally occurring C5R1, and all such variations are to be considered as being specifically disclosed.

Although the nucleotide sequences which encode a C5R1, its derivatives or its variants are preferably capable of hybridizing to the nucleotide sequence of the naturally occurring C5AR1 polynucleotide under stringent conditions, it may be advantageous to produce nucleotide sequences encoding C5aR1 polypeptides or its derivatives possessing a substantially different codon usage. Codons can be selected to increase the rate at which expression of the peptide occurs in a particular prokaryotic or eukaryotic expression host in accordance with the frequency with which particular codons are utilized by the host. Other reasons for substantially altering the nucleotide sequence encoding a C5aR1 polypeptide and/or its derivatives without altering the encoded amino acid sequence include the production of RNA transcripts having more desirable properties, such as a greater half-life, than transcripts produced from the naturally occurring sequence.

Nucleotide sequences encoding a C5aR1 polypeptide may be joined to a variety of other nucleotide sequences by means of well established recombinant DNA techniques. Useful nucleotide sequences for joining to C5aR1 polynucleotides include an assortment of cloning vectors such as plasmids, cosmids, lambda phage derivatives, phagemids, and the like. Vectors of interest include expression vectors, replication vectors, probe generation vectors, sequencing vectors, etc. In general, vectors of interest may contain an origin of replication functional in at least one organism, convenient restriction endonuclease sensitive sites, and selectable markers for one or more host cell systems.

Another aspect of the subject invention is to provide for C5aR1-specific hybridization probes capable of hybridizing with naturally occurring nucleotide sequences encoding C5aR1. Such probes may also be used for the detection of similar protease encoding sequences and should preferably show at least 40% nucleotide identity to C5aR1 polynucleotides. The hybridization probes of the subject invention may be derived from the nucleotide sequence presented as SEQ ID NO: 1 or from genomic sequences including promoter, enhancers or introns of the native gene. Hybridization probes may be labelled by a variety of reporter molecules using techniques well known in the art.

It will be recognized that many deletional or mutational analogs of C5aR1 polynucleotides will be effective hybridization probes for C5aR1 polynucleotides. Accordingly, the invention relates to nucleic acid sequences that hybridize with such C5aR1 encoding nucleic acid sequences under stringent conditions.

“Stringent conditions” refers to conditions that allow for the hybridization of substantially related nucleic acid sequences. For instance, such conditions will generally allow hybridization of sequence with at least about 85% sequence identity, preferably with at least about 90% sequence identity, more preferably with at least about 95% sequence identity, or most preferably with at least about 99% sequence identity. Hybridization conditions and probes can be adjusted in well-characterized ways to achieve selective hybridization of human-derived probes. Stringent conditions, within the meaning of the invention are 65° C. in a buffer containing 1 mM EDTA, 0.5 M NaHPO4 (pH 7.2), 7% (w/v) SDS.

Nucleic acid molecules that will hybridize to C5aR1 polynucleotides under stringent conditions can be identified functionally. Without limitation, examples of the uses for hybridization probes include: histochemical uses such as identifying tissues that express C5aR1; measuring mRNA levels, for instance to identify a sample's tissue type or to identify cells that express abnormal levels of C5aR1; and detecting polymorphisms of C5aR1.

PCR provides additional uses for oligonucleotides based upon the nucleotide sequence which encodes C5aR1. Such probes used in PCR may be of recombinant origin, chemically synthesized, or a mixture of both. Oligomers may comprise discrete nucleotide sequences employed under optimized conditions for identification of C5AR1 in specific tissues or diagnostic use. The same two oligomers, a nested set of oligomers, or even a degenerate pool of oligomers may be employed under less stringent conditions for identification of closely related DNAs or RNAs. Rules for designing polymerase chain reaction (PCR) primers are now established, as reviewed by PCR Protocols. Degenerate primers, i.e., preparations of primers that are heterogeneous at given sequence locations, can be designed to amplify nucleic acid sequences that are highly homologous to, but not identical with C5AR1. Strategies are now available that allow for only one of the primers to be required to specifically hybridize with a known sequence. For example, appropriate nucleic acid primers can be ligated to the nucleic acid sought to be amplified to provide the hybridization partner for one of the primers. In this way, only one of the primers need be based on the sequence of the nucleic acid sought to be amplified.

PCR methods for amplifying nucleic acid will utilize at least two primers. One of these primers will be capable of hybridizing to a first strand of the nucleic acid to be amplified and of priming enzyme-driven nucleic acid synthesis in a first direction. The other will be capable of hybridizing the reciprocal sequence of the first strand (if the sequence to be amplified is single stranded, this sequence will initially be hypothetical, but will be synthesized in the first amplification cycle) and of priming nucleic acid synthesis from that strand in the direction opposite the first direction and towards the site of hybridization for the first primer. Conditions for conducting such amplifications, particularly under preferred stringent hybridization conditions, are well known. Other means of producing specific hybridization probes for C5aR1 include the cloning of nucleic acid sequences encoding C5aR1 or C5aR1 derivatives into vectors for the production of mRNA probes. Such vectors are known in the art, are commercially available and may be used to synthesize RNA probes in vitro by means of the addition of the appropriate RNA polymerase as T7 or SP6 RNA polymerase and the appropriate reporter molecules.

It is possible to produce a DNA sequence, or portions thereof, entirely by synthetic chemistry. After synthesis, the nucleic acid sequence can be inserted into any of the many available DNA vectors and their respective host cells using techniques which are well known in the art.

Moreover, synthetic chemistry may be used to introduce mutations into the nucleotide sequence. Alternately, a portion of sequence in which a mutation is desired can be synthesized and recombined with longer portion of an existing genomic or recombinant sequence.

C5aR1 polynucleotides may be used to produce a purified oligo- or polypeptide using well known methods of recombinant DNA technology. The oligopeptide may be expressed in a variety of host cells, either prokaryotic or eukaryotic. Host cells may be from the same species from which the nucleotide sequence was derived or from a different species. Advantages of producing an oligonucleotide by recombinant DNA technology include obtaining adequate amounts of the protein for purification and the availability of simplified purification procedures.

The C5aR1 receptor antagonist W-54011 is defined by CAS number: 405098-33-1.

### Identification of Hamster C5a Receptor 1

### Species Differences

Human C5a is a 74 amino acid peptide which contains an N-linked carbohydrate moiety attached to Asn64. This glycosylation is not necessary for full biological activity in vitro, but may be involved in modulating C5a activity in vivo. The solution structure of human C5a has been determined by NMR spectroscopy and consists of a disulfide-linked core segment (1-63) and a disordered C-terminal segment (64-74). Recently, using a different set of solvent conditions, an a-helical conformation was found for the residues 69-74 with a short loop connecting this helix to the core domain bringing Arg74 close to Arg62. The relevance of this particular solution structure to the receptor-bound conformation of C5a is not known. A two-site model for the binding of C5a to its receptor has been proposed [22]. The chief ‘binding domain’ (Site 1) is located in the extracellular N-terminus of the membrane spanning receptor and interacts with the 4-helix bundle core of C5a. The ‘activating domain’ (Site 2) binds the C-terminal 8 amino acids of C5a and appears to lie in or near the receptor's interhelical region. This theory has some support from site-directed mutagenesis studies which have identified particular residues in both C5a and its receptor that are associated with biological activity [22]. Since the interaction of C5a with its receptor appears to involve two major sites or domains, C5a itself can be considered to be composed of two regions, a short C-terminal activation domain of about 10 residues, and a longer N-terminal helical bundle receptor-binding domain of 64 residues. In principle, an antagonist molecule would only need to block one of these key interacting regions of C5a to prevent activation of the C5a receptor (C5aR). Antagonists to both sites have been obtained through synthesis of peptide analogs of C5a and by random screening of compound libraries. Antagonists of C5a can be classified according to their size as proteins, small peptides or small non-peptidic compounds.

To date, C5aR has been cloned from human, rat, mouse, dog, rabbit, guinea pig, pig, sheep and several non-human primates (partial). Interestingly, C5aR sequence homology across these various species is unusually divergent. Overall C5aR sequence homology is 95% between human and non-human primate. Conversely, between human and non-primate C5aR5, homology is only 65-75%. These differences are unusual for G-protein-coupled receptors, which are typically 85-95% homologous across species. All full-length, recombinant and natively expressed C5aR5, except rat, bind human C5a with high affinity, suggesting relative conservation of C5a ligand-binding domains. However, cyclic peptide and small molecule C5aR antagonists demonstrate a greater degree of species selectivity. This suggests different C5aR binding and activation determinants for C5a peptide and small molecule antagonists. A small molecule C5aR antagonist (W-54011) inhibits C5a-mediated responses in human, cynomolgus monkey, and gerbil neutrophils, but not in mouse, rat, guinea pig, rabbit, or dog neutrophils. Because of this observed small molecule antagonist species selectivity could be responsible for the observed species-selective pharmacology [23].

An object if the invention is a C5aR1 polynucleotide, selected from a group consisting of

(i) nucleic acid molecules encoding a polypeptide comprising the amino acid sequence of SEQ ID NO: 2,
(ii) nucleic acid molecules comprising the sequence of SEQ ID NO: 1,
(iii) nucleic acid molecules having the sequence of SEQ ID NO: 1,
(iv) nucleic acid molecules the complementary strand of which hybridizes under stringent conditions to a nucleic acid molecule of (i), (ii), or (iii); and
(v) nucleic acid molecules the sequence of which differs from the sequence of a nucleic acid molecule of (iii) due to the degeneracy of the genetic code;
wherein the polypeptide encoded by said nucleic acid molecule has C5aR1 activity.

A further object if the invention is a C5aR polypeptide selected from a group consisting of

(i) polypeptides having the sequence of SEQ ID NO: 2,
(ii) polypeptides comprising the sequence of SEQ ID NO: 2,
(iii) polypeptides encoded by C5aR1 polynucleotides as disclosed above; and
(iv) polypeptides which have at least 85%, 90%, 95%, 98% or 99% identity,
wherein said polypeptide has C5aR1 activity.

In a more preferred object of the invention the aforementioned polypeptides have C5aR1 activity, which is inhibitable by the antagonist W-54011. It is an aspect of the invention to provide a non-human, non-primate new C5aR1 polypeptide which activation by C5a ligand is antagonizeable or inhibitable by the C5aR1 antagonist W-54011. Inhibition or antagonization by W-54011 is at least 10%, 20%, 30%, 50%, 60%, 70%, 80%, 90% or 95%. Preferably, the inhibition is at least 30%. The inhibition of the C5aR1 activity by W-54011 provides a polypeptide which is human-like and therefore suitable for pharmacological studies of C5aR1 modulators as shown for example in FIGS. 6-11.

### Inhibitors of the Complement System

The complement system is important for the host defense against infectious pathogens and serves to initiate the inflammatory response. The complement system directly kills and promotes the phagocytosis of invading microorganisms, it facilitates the primary and secondary antibody responses of B cells and effects the clearance of immune complexes. Thirty plasma and membrane components, factors, regulators and receptors of the complement system are linked in biochemical cascades, named classical, alternative and lectin pathways. The involvement of this system in the early phases of the inflammatory response, as well as the wide array of proinflammatory consequences of complement activation, makes the complement system an attractive target for therapeutic intervention and has led to the isolation, design and synthesis of numerous complement inhibitors. Activation of the complement system leading to disease complications often arises from incomplete biocompatibility of materials of apparatuses for hemodialysis, artificial hearts and other facilities. As complement activation is a significant factor in allograft rejection and eventually for long-time graft survival, the application of complement inhibitors is necessary in allotransplantology. Hyperacute rejection of xenografts can also be prevented by complement blocking compounds. To date, however, no specific complement inhibitors have been approved for clinical use.

### Animal Models

The human genome contains ˜30,000 genes that could encode >1,000,000 different proteins via RNA editing, alternative splicing, and post-translational modifications. To date, only 500 gene products have been identified as molecular drug targets to treat human illnesses. A theoretical number of at least 5,000-15,000 potential gene products (or molecular drug targets) have been proposed that could lead to more effective or selective therapies. The pharmaceutical industry and biotechnology companies are now heavily focussed on using tools that can provide a better understanding of the function or product of a gene, and that enable the rapid identification and validation of a human drug target among numerous potential candidates. Potential therapeutics could be not only small chemical drug molecules that modulate the function of a protein but also the gene products themselves. The use of phylogenetically lower model organisms to mimic human diseases has become very popular as it enables either the identification of a human gene product (or pathway) that is directly involved in a disease state, or the development of biological screens for molecules or gene products that suppress the disease or stop its progression. The mouse, despite its very low throughput, remains the organism of choice for many close functional parallels with human diseases [24]. For complement related diseases and processes it is necessary to use specific animal models due to the known species-selective pharmacology.

We have identified hamster as a species which could be used as an animal model for the characterization of complement system modulators within the drug discovery process. Surprisingly the hamster C5aR1 receptor has the same critical amino acids which are necessary for specific modulator activity. Therefore the use of hamster it not limited to the use as an animal model for the characterization of C5aR1 modulators, but also for the characterization of complement modulators itself.

### Complement Model—Cecal Ligation and Puncture (CLP)

Sepsis and multi organ failure are the most important cause of death among hospitalized patients, with mortality rates ranging from 30 to 70%. Despite advances in supportive care, each year 750,000 people develop sepsis and 225,000 die in the United States alone, and the incidence of sepsis is rising at rates between 1.5% and 8% per year. Sepsis is the result of an acute and systemic immune response to a variety of noxious insults, in particular to bacterial infection. This response leads to the activation of a number of host mediator systems, including the cytokine, leukocyte, and hemostatic networks, each of which may contribute to the pathological sequelae of sepsis. Bacterial sepsis can be induced by cecal ligation puncture (CLP) which induces multiorgan failure [25]. CLP offers a stable model for sepsis mimicking the human situation where colon perforation results in peritonitis which is a common cause for sepsis. CLP sepsis models are described in mice and rats but so far not in gerbils. Therefore, we performed pilot studies to establish the CLP sepsis model in hamster.

The described CLP model could be used to characterize C5aR1 modulations in vivo. We have identified biomarkers which are useful to monitor indirectly the activity of C5aR1 modulators in lung failure, kidney failure, heart failure and multi organ failure.

In FIG. 6 it is shown that kidney failure or disorders leads to an increase of IL10 expression in kidney tissue. The inhibition of C5aR1 leads dose dependently to a reversal to this effect. The expression of IL 10 is decreased under C5aR1 modulator treatment compared to untreated animals (CLP+vehicle vs. CLP+0.5 mg/kg and CLP+2.5 mg/kg and CLP+Alzet). Here we show that the modulation of C5aR1 in a hamster model leads to kidney protection.

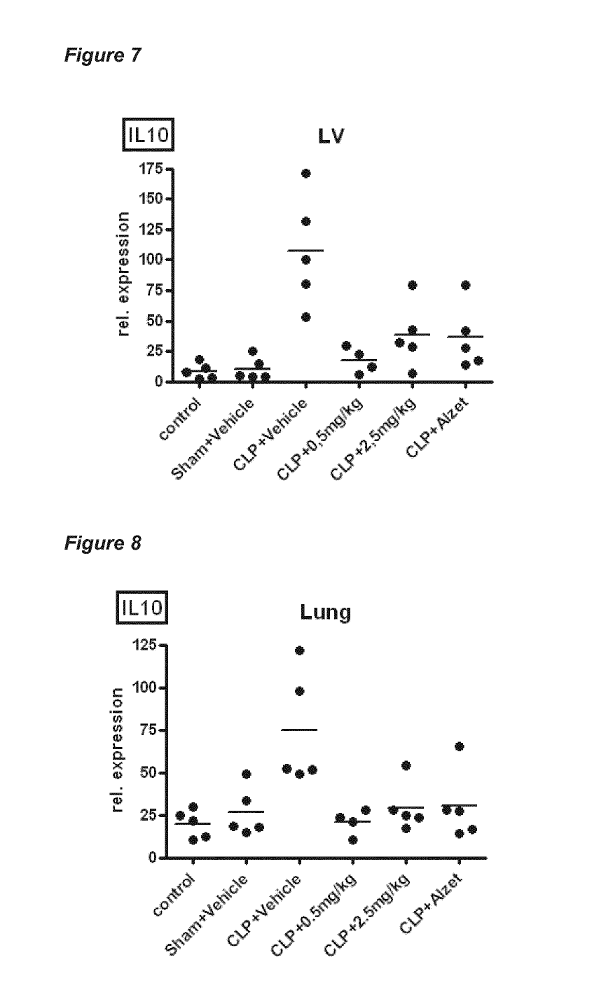

In FIG. 7 it is shown that heart failure (LV: left ventricle/heart) or disorders leads to an increase of IL10 expression in LV tissue. The inhibition of C5aR1 leads dose dependently to a reversal to this effect. The expression of IL10 is decreased under C5aR1 modulator treatment compared to untreated animals (CLP+vehicle vs. CLP+0.5 mg/kg and CLP+2.5 mg/kg and CLP+Alzet). Here we show that the modulation of C5aR1 in a hamster model leads to heart protection.

In FIG. 8 it is shown that lung failure or disorders leads to an increase of IL10 expression in lung tissue. The inhibition of C5aR1 leads dose dependently to a reversal to this effect. The expression of IL10 is decreased under C5aR1 modulator treatment compared to untreated animals (CLP+vehicle vs. CLP+0.5 mg/kg and CLP+2.5 mg/kg and CLP+Alzet). Here we show that the modulation of C5aR1 in a hamster model leads to lung protection.

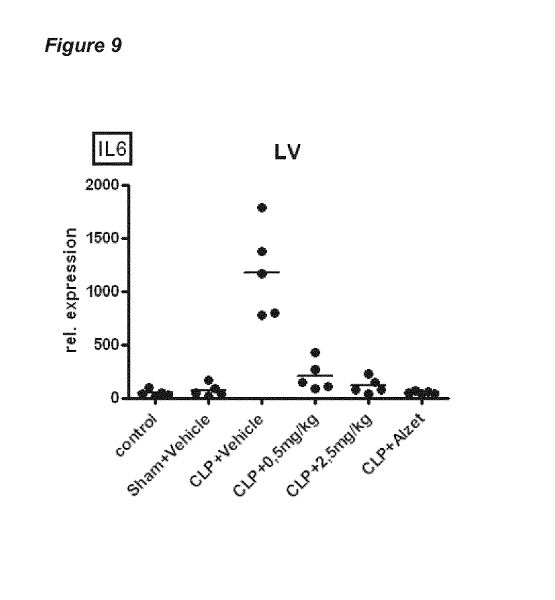

In FIG. 9 it is shown that heart failure (LV: left ventricle/heart) or disorders leads to an increase of IL6 expression in LV tissue. The inhibition of C5aR1 leads dose dependently to a reversal to this effect. The expression of IL6 is decreased under C5aR1 modulator treatment compared to untreated animals (CLP+vehicle vs. CLP+0.5 mg/kg and CLP+2.5 mg/kg and CLP+Alzet). Here we show that the modulation of C5aR1 in a hamster model leads to heart protection.

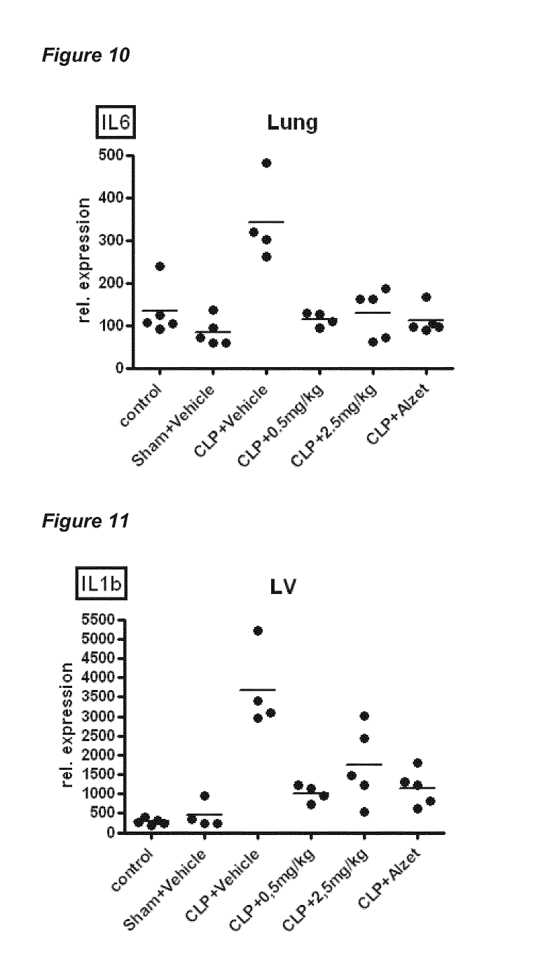

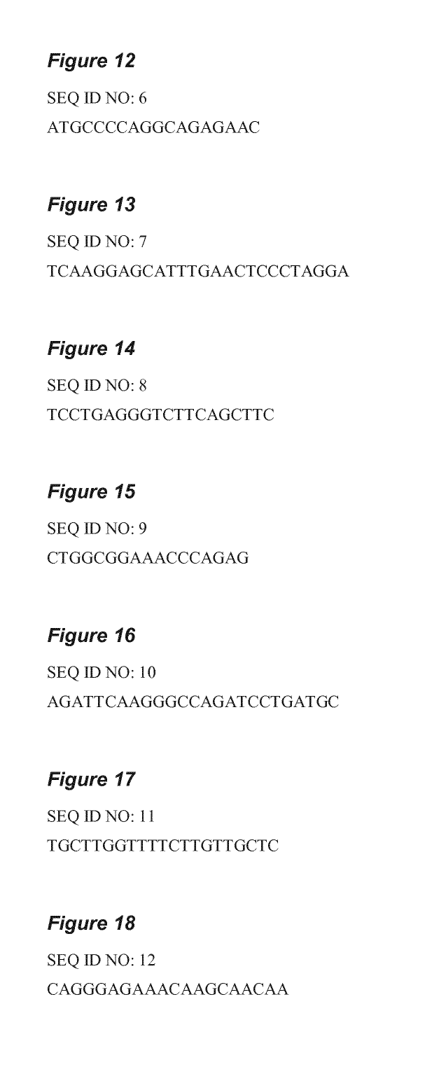

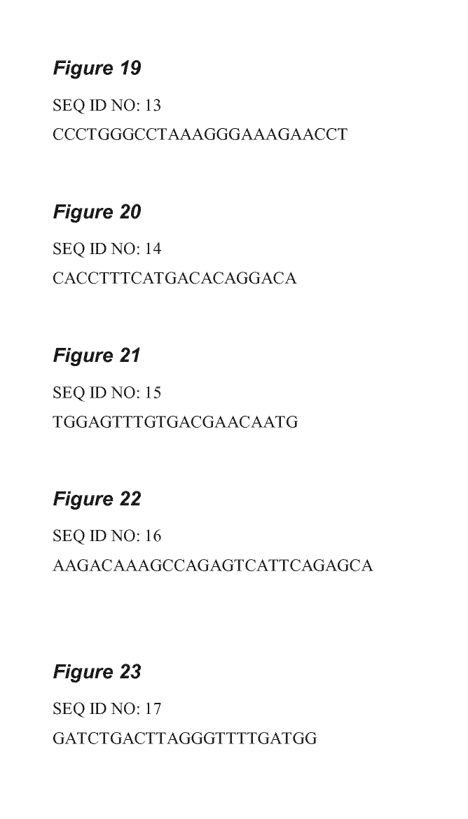

In FIG. 10 it is shown that lung failure or disorders leads to an increase of IL6 expression in lung tissue. The inhibition of C5aR1 leads dose dependently to a reversal to this effect. The expression of IL6 is decreased under C5aR1 modulator treatment compared to untreated animals (CLP+vehicle vs. CLP+0.5 mg/kg and CLP+2.5 mg/kg and CLP+Alzet). Here we show that the modulation of C5aR1 in a hamster model leads to lung protection.

In FIG. 11 it is shown that heart failure (LV: left ventricle/heart) or disorders leads to an increase of IL1b expression in LV tissue. The inhibition of C5aR1 leads dose dependently to a reversal to this effect. The expression of IL1b is decreased under C5aR1 modulator treatment compared to untreated animals (CLP+vehicle vs. CLP+0.5 mg/kg and CLP+2.5 mg/kg and CLP+Alzet). Here we show that the modulation of C5aR1 in a hamster model leads to heart protection.

Surprisingly we have identified hamster as an animal model which could be used to characterize C5aR1, C5a and C5 modulators in vitro and in vivo. Hamster could be used to characterize those modulators in kidney failure or disorders, heart failure or disorders, lung failure or disorders and multi organ failure or dysfunction. IL6, IL10 and IL1b, but not limited to, could be used as biomarker to monitor organ damage or function.

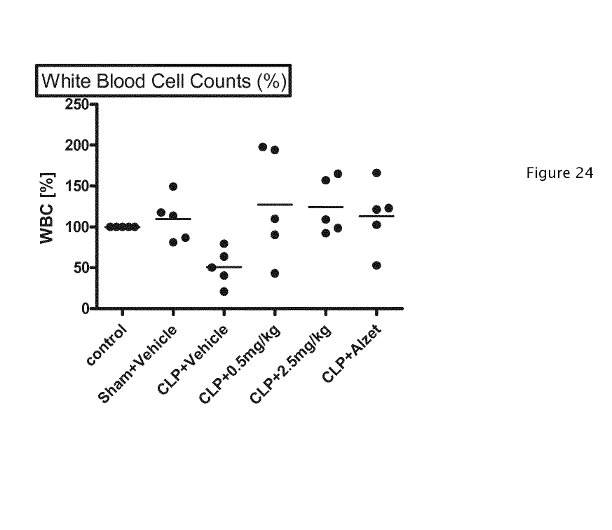

In FIG. 24 it is shown that the used CLP hamster model leads to hematologic effects. The WBC is decreased under CLP (without treatment). The treatment of CLP hamster with C5aR1 modulators leads to a reversion of those effects. The WBC of sham or control animal is higher compared to the WBC of CLP+vehicle. The WBC of CLP+0.5 mg/kg and CLP+2.5 mg/kg and CLP+Alzet is higher compared to CLP+vehicle. The treatment of hamster with kidney failure or lung failure or heart failure or multi-organ failure with C5aR1 modulators (here inhibitors) are leading to multi organ protection and normalization of WBC.

The WBC count, but not limited to, could be used a biomarker to monitor the disease stage and efficacy of C5aR1 modulators. Furthermore other physiological, biochemical, phenotypic and molecular biomarkers could be used to monitor the efficacy of C5aR1, C5a or C5 modulators in hamster. The described. CLP model is used as an example for organ failure, inflammation or complement system activating models in hamster. Hamster could be used for the characterization of C5aR1 modulators for all models which show activation of the complement system. The activation of the complement system could be shown by measuring the different members as C5, C5a, C3 and so forth by biochemical or molecular methods. These parameters could be used to select the appropriate hamster model.

The term “hamster” within the meaning of the invention describes the mammal family of Cricetinae. In a preferred object of the invention the Cricetinae is selected from the group consisting of Allocricetulus, Cansumys, Cricetulus, Cricetus, Mesocricetus, Phodopus and Tscherskia. In a even more preferred object the Cricetinae is a Mesocricetus auratus (also named Golden or Syrian Hamster).

### Biomarkers for Complement System Activation and Modulators

### Classes:

Disease Biomarker: a biomarker that relates to a clinical outcome or measure of disease.

Efficacy Biomarker: a biomarker that reflects beneficial effect of a given treatment.

Staging Biomarker: a biomarker that distinguishes between different stages of a chronic disorder.

Surrogate Biomarker: a biomarker that is regarded as a valid substitute for a clinical outcomes measure.

Toxicity Biomarker: a biomarker that reports a toxicological effect of a drug on an in vitro or in vivo system.

Mechanism Biomarker: a biomarker that reports a downstream effect of a drug.

Target Biomarker: a biomarker that reports interaction of the drug with its target.

### Expression Analysis

### IL10

Interleukin-10 (IL-10 or IL10), also known as human cytokine synthesis inhibitory factor (CSIF), is an anti-inflammatory cytokine. In humans IL-10 is encoded by the IL10 gene. This cytokine is produced primarily by monocytes and to a lesser extent by lymphocytes. This cytokine has pleiotropic effects in immunoregulation and inflammation. It down-regulates the expression of Th1 cytokines, MHC class II antigens, and co-stimulatory molecules on macrophages. It also enhances B cell survival, proliferation, and antibody production. This cytokine can block NF-κB activity, and is involved in the regulation of the JAK-STAT signaling pathway. Knockout studies in mice suggested the function of this cytokine as an essential immunoregulator in the intestinal tract and indeed patients with Crohn's disease react favorably towards treatment with bacteria producing recombinant interleukin 10, showing the importance of interleukin 10 for counteracting excessive immunity in the human body. A study in mice has shown that interleukin-10 is also produced by mast cells, counteracting the inflammatory effect that these cells have at the site of an allergic reaction. It is capable of inhibiting synthesis of pro-inflammatory cytokines like IFN-γ, IL-2, IL-3, INFα and GM-CSF made by cells such as macrophages and regulatory T-cells. IL-10 also displays potent abilities to suppress the antigen presentation capacity of antigen presenting cells. However, it is also stimulatory towards certain T cells, mast cells and stimulates B cell maturation and antibody production. It is mainly expressed in monocytes and Type 2 T helper cells (TH2), mast cells, CD4+CD25+Foxp3+ regulatory T cells, and also in a certain subset of activated T cells and B cells. Said et al. showed that IL-10 can also be produced by monocytes upon PD-1 triggering in this cells.

An increase of IL10 expression or protein level indicates a systemic or local inflammatory process and could be used as a biomarker. The IL10 expression level is elevated in different tissues from hamster CLP model. An anti-complement treatment, as C5aR1 inhibition leads to a normalization of the IL10 expression level in hamster, as shown on FIGS. 6 to 8.

### IL6

Interleukin-6 (IL-6) is a protein that in humans is encoded by the IL6 gene. IL-6 is an interleukin that acts as both a pro-inflammatory and anti-inflammatory cytokine. It is secreted by T cells and macrophages to stimulate immune response to trauma, especially burns or other tissue damage leading to inflammation. In terms of host response to a foreign pathogen, IL-6 has been shown, in mice, to be required for resistance against the bacterium, Streptococcus pneumoniae. IL-6 is also a “myokine,” a cytokine produced from muscle, and is elevated in response to muscle contraction. It is significantly elevated with exercise, and precedes the appearance of other cytokines in the circulation. During exercise, it is thought to act in a hormone-like manner to mobilize extracellular substrates and/or augment substrate delivery. Additionally, osteoblasts secrete IL-6 to stimulate osteoclast formation. Smooth muscle cells in the tunica media of many blood vessels also produce IL-6 as a pro-inflammatory cytokine. IL-6's role as an anti-inflammatory cytokine is mediated through its inhibitory effects on TNF-alpha and IL-1, and activation of IL-1ra and IL-10. IL-6 is one of the most important mediators of fever and of the acute phase response. It is capable of crossing the blood brain barrier and initiating synthesis of PGE2 in the hypothalamus, thereby changing the body's temperature setpoint. In the muscle and fatty tissue, IL-6 stimulates energy mobilization which leads to increased body temperature. IL-6 can be secreted by macrophages in response to specific microbial molecules, referred to as pathogen associated molecular patterns (PAMPs). These PAMPs bind to a highly important group of detection molecules of the innate immune system, called pattern recognition receptors (PRRs), including Toll-like receptors (TLRs). These are present on the cell surface and intracellular compartments and induce intracellular signaling cascades that give rise to inflammatory cytokine production. IL-6 is also essential for hybridoma growth and is found in many supplemental cloning media such as briclone. Inhibitors of IL-6 (including estrogen) are used to treat postmenopausal osteoporosis. Il-6 is also produced by adipocytes and is thought to be a reason why obese individuals have higher endogenous levels of CRP. In a 2009 study, intranasally administered IL-6 was shown to improve sleep-associated consolidation of emotional memories.

An increase of IL6 expression or protein level indicates a systemic or local inflammatory process and could be used as a biomarker. The IL6 expression level is elevated in different tissues from hamster CLP model. An anti-complement treatment, as C5aR1 inhibition leads to a normalization of the IL6 expression level in hamster, as shown on FIGS. 9 and 10.

### IL1b

Interleukin-1 beta (IL-1β) also known as catabolin, is a cytokine protein that in humans is encoded by the IL1B gene. IL-1β precursor is cleaved by caspase 1 (interleukin 1 beta convertase). UL-1β is a member of the interleukin 1 cytokine family. This cytokine is produced by activated macrophages as a proprotein, which is proteolytically processed to its active form by caspase 1 (CASP1/ICE). This cytokine is an important mediator of the inflammatory response, and is involved in a variety of cellular activities, including cell proliferation, differentiation, and apoptosis. The induction of cyclooxygenase-2 (PTGS2/COX2) by this cytokine in the central nervous system (CNS) is found to contribute to inflammatory pain hypersensitivity.

An increase of IL1b expression or protein level indicates a systemic or local inflammatory process and could be used as a biomarker. The IL1b expression level is elevated in different tissues from hamster CLP model. An anti-complement treatment, as C5aR1 inhibition leads to a normalization of the IL1b expression level in hamster, as shown on FIG. 11.

### Hemogram

Blood samples were obtained under light Isoflurane anesthesia from the cavernous sinus with a capillary at different time points/final exsanguination by cannulation of the carotid artery after 24 hr to allow measurements of differential blood counts. Blood samples for basal blood cell counts were collected from the cavernous sinus one week before study begin. Blood was collected into EDTA tubes and blood cell counts were performed on an automated cell counter.

### Structure-Based Inhibitor Design

The molecular cloning and biochemical analysis of many components of the complement system during the past two decades have led to a detailed understanding of the mechanisms of complement activation. Determinations of 3D structures of many complement components and their binding sites triggered new efforts in the complement inhibitors field. The classical complement pathway is usually activated when component C1q binds to a complex of antigen and IgM or IgG antibody. It was established that C1q binding site on IgG resides in the CH2 domain. Several groups have proposed different regions as possible complement binding sites and obtained polypeptides resembling these sequences. These synthetic peptides bind to C1q and prevent its interaction with antibodies. Using this approach, several selective inhibitors of the first component of the complement system that inhibit only the classical pathway of complement activation have been obtained. Trp277 and Tyr278 residues of the CH2 domain of immunoglobulin have been determined to be involved in C1q-IgG interaction. Considering that C1q has six globular heads, each with one or more binding site(s) for immunoglobulin, Anderson et al. Positively charged amidine group of compound (xii) forms a salt bridge with the negatively charged Asp residue of C1s with the thiophene ring fully occupying the binding pocket. Molecular modifications of the lead thiophenamidine (xii) have led to the construction of a novel series of potent and selective inhibitors of human C1s. Compound (xiii) is one of them (IC50=0.300 μM).

Inhibitors Resulting from Phage Display

A series of inhibitors of the complement system was revealed by phage display, a method based on expressing recombinant proteins or peptides fused to a phage coat protein. Phage display is a very powerful technique for obtaining libraries containing millions or even billions of different peptides or proteins. It is used to identify ligands for peptide receptors, define epitopes for monoclonal antibodies, and select enzyme substrates. Compstatin was isolated from a phage-displayed random peptide library as a ligand of complement component C3. This peptide has a cyclic structure consisting of 13 amino acid residues (ICVVQDWGHHRCT-NH2, IC50=12 μM). In a series of experiments, compstatin was shown to inhibit complement activation in human serum and heparine and protamine-induced complement activation in primates without significant side effects. It prolongs the lifetime of a porcine-to-human xenograft perfused with human blood and inhibits complement activation in many models of complement-mediated diseases. It is reported that the sequences of 42 peptides that were selected from phage display libraries on the basis of binding to protein C1q. From peptides that showed inhibition of C1q hemolytic activity but no inhibition of the alternative complement pathway, one cyclic peptide 2J (CEGPFGPRHDLTFCW) was selected and studied. This peptide has promising properties for therapeutic complement inhibition because it specifically inhibits the classical complement pathway (IC50=2-6 μM) at the earliest possible level, preventing anaphylactic reactions of C3a, C4a and C5a [4].

### High Molecular Weight Natural Inhibitors

Under physiological conditions, complement activation is regulated by a series of membrane-bound and soluble complement control proteins. It has been recognized that some of the endogenous complement regulatory proteins might serve as potential therapeutic agents in blocking inappropriate activation of complement in human diseases. A soluble version of recombinant human CR1 (sCR1) lacking the transmembrane and cytoplasmic domains was produced and shown to retain all the known functions of the native CR1. sCR1 has been shown to reduce complement-mediated tissue injury in models of ischemia-reperfusion and animal models of a wide range of human acute and chronic inflammatory diseases (dermal vascular reactions, lung injury, trauma, myasthenia gravis, glomerulonephritis, multiple sclerosis, allergic reactions and asthma). Unfortunately, sCR1 has a short half-life in circulation. A longer half-life would permit bolus administration, allow lower doses of the drug to achieve comparable therapeutic effects and reduce the cost per therapeutic dosage. To prolong the half-life of sCR1, the protein was obtained as a fusion protein with albumin-binding terminus of Streptococcal protein G. Chimeric molecules based on functional fragments of CR1 and IgG not only have a longer half-life, but might also act as complement inhibitors in specific tissues. Inhibition of C5 activation using high-affinity anti-C5 monoclonal antibodies represents another therapeutic approach for blocking complement activation. This strategy is aimed at inhibiting the formation of C5a and C5b-9 via the classical and alternative pathways, without affecting the generation of C3b, which is critical for antibacterial defense. Although monoclonal antibodies could be used in human therapy, it is recognized that chronic application of monoclonal antibodies would elicit human anti-mouse antibody responses. The ‘humanization’ of antibodies minimizes immunogenic reactions, although it might be difficult to completely eliminate anti-idiotypic effects. Recent advances in transgenic animal technology make it possible to produce completely human monoclonal antibodies devoid of mouse or other nonhuman sequences. At present, PEGylation (conjugation of proteins with PEG molecules) is used to increase the half-life in circulation, reduce immunogenicity and prevent proteolytic inactivation. These effects are due to a shell of PEG molecules around the protein that sterically hinders the reactions with immune cells [4].

### Indications

### Sepsis

Sepsis is a potentially deadly medical condition that is characterized by a whole-body inflammatory state (called a systemic inflammatory response syndrome or SIRS). The body may develop this inflammatory response by the immune system to microbes in the blood, urine, lungs, skin, or other tissues. Severe sepsis is the systemic inflammatory response and could be combined with infection and organ dysfunction.

Severe sepsis is usually treated in the intensive care unit with intravenous fluids and antibiotics. If fluid replacement is insufficient to maintain blood pressure, specific vasopressor medications can be used. Mechanical ventilation and dialysis may be needed to support the function of the lungs and kidneys, respectively. To guide therapy, a central venous catheter and an arterial catheter may be placed; measurement of other hemodynamic variables (such as cardiac output, or mixed venous oxygen saturation) may also be used. Sepsis patients require preventive measures for deep vein thrombosis, stress ulcers and pressure ulcers, unless other conditions prevent this.

The immunological response causes widespread activation of acute-phase proteins, affecting the complement system and the coagulation pathways, which could cause damage to the vasculature as well as to the organs. Various neuroendocrine counter-regulatory systems are then activated as well, often compounding the problem. Even with immediate and aggressive treatment, this may progress to multiple organ dysfunction syndrome and eventually death.

### Heart Failure

Cardiac failure is a condition in which the output of the heart is not adequate to meet the needs of the body, either at rest or with exercise. This is usually accompanied by an increased filling pressure and/or volume. The condition requires prompt recognition and management since tissue oxygen supply and hence organ function can both be readily compromised. Congestive heart failure is the presence of heart failure and oedema in the presence of normal systolic function. In these patients, it is important to exclude other diseases such as valvular disease, recurrent ischaemia, pericardial disease, cor pulmonale and congenital heart disease as the cause of congestive heart failure. Often, these conditions arise because of diastolic dysfunction. Acute heart failure is not a single entity, occurring during diastole or systole. To determine the type of cardiac failure, it is necessary to understand the normal physiology and the factors, which regulate myocardial contraction.

Ventricular function is decreased during sepsis. Patients with septic shock have been documented to have lowered ejection fractions—mean of 32%—despite an increase in cardiac output. This returned to normal within 10 days in survivors. Similar findings have been observed in human volunteers given endotoxin. Ejection fraction is not a pure measure of systolic contractility of the heart but is a measure of ventricular function which also depends on diastolic compliance, preload and afterload.

### Lung Failure (Respiratory Failure, ARDS, ALI)

The term respiratory failure is used to describe inadequate gas exchange by the respiratory system, with the result that arterial oxygen and/or carbon dioxide levels cannot be maintained within their normal ranges. A drop in blood oxygenation is known as hypoxemia; arise in arterial carbon dioxide levels is called hypercapnia. The normal reference values are: oxygen PaO2 greater than 80 mmHg (11 kPa), and carbon dioxide PaCO2 less than 45 mmHg (6.0 kPa). Classification into type I or type II relates to the absence or presence of hypercapnia respectively.

Acute respiratory distress syndrome (ARDS), also known as respiratory distress syndrome (RDS) or adult respiratory distress syndrome (in contrast with IRDS) is a serious reaction to various forms of injuries to the lung. ARDS is a severe lung disease caused by a variety of direct and indirect issues. It is characterized by inflammation of the lung parenchyma leading to impaired gas exchange with concomitant systemic release of inflammatory mediators causing inflammation, hypoxemia and frequently resulting in multiple organ failure. This condition is often fatal, usually requiring mechanical ventilation and admission to an intensive care unit. A less severe form is called acute lung injury (ALI).

### Renal Failure

Renal failure or kidney failure describes a medical condition in which the kidneys fail to adequately filter toxins and waste products from the blood. The two forms are acute (acute kidney injury) and chronic (chronic kidney disease); a number of other diseases or health problems may cause either form of renal failure to occur. Renal failure is described as a decrease in the glomerular filtration rate. Biochemically, renal failure is typically detected by an elevated serum creatinine level. Problems frequently encountered in kidney malfunction include abnormal fluid levels in the body, deranged acid levels, abnormal levels of potassium, calcium, phosphate, and (in the longer term) anemia as well as delayed healing in broken bones. Depending on the cause, hematuria (blood loss in the urine) and proteinuria (protein loss in the urine) may occur.

### SIRS

In medicine, systemic inflammatory response syndrome (SIRS) is an inflammatory state affecting the whole body, frequently a response of the immune system to infection, but not necessarily so. It is related to sepsis, a condition in which individuals both meet criteria for SIRS and have a known or highly suspected infection. SIRS is a serious condition related to systemic inflammation, organ dysfunction, and organ failure. It is a subset of cytokine storm, in which there is abnormal regulation of various cytokines.

The SIRS is defined as a disease which is associated with the multiple (rather than a single) etiologies associated with organ dysfunction and failure following a hypotensive shock episode. The active pathways leading to such pathophysiology may include fibrin deposition, platelet aggregation, coagulopathies and leukocyte liposomal release. The implication of such a definition suggests that recognition of the activation of one such pathway is often indicative of that additional pathophysiologic processes are also active and that these pathways are synergistically destructive. The clinical condition may lead to renal failure, respiratory distress syndrome, central nervous system dysfunction and possible gastrointestinal bleeding.

SIRS is frequently complicated by failure of one or more organs or organ systems. The complications of SIRS include (but not limited to): acute lung injury, acute kidney injury, multiple organ dysfunction syndrome. [26, 27, 28]

### Use of Hamster Genes of the Complement System for In Vitro and In Vivo Testing

One embodiment of the invention is a method to use genes from the hamster complement system to characterize human complement system modulators comprising:
(a) activators of C5 or C5a
(b) modulators of C5 activity
(c) modulators of C5a activity
(d) modulators of C5aR1 activity

One embodiment of the invention is a method to use the hamster complement system to characterize human complement system modulators in vivo comprising:
(a) models for sepsis
(b) models for SIRS
(c) models for organ dysfunction
(d) models for neurodegenerative diseases
(e) models for heart failure
(f) models for renal failure
(g) models for lung failure
(h) models for systemic inflammation

One embodiment of the invention is a method to use polypeptides from the hamster complement system to characterize human complement system modulators in vitro comprising:
(a) binding assays
(b) activity assays
(c) clinical chemistry parameters.

In a preferred object of the invention the human complement system modulator is a C5aR1 modulator, even further preferred is a C5aR1 antagonist or inhibitor.

One embodiment of the invention is a method to use genes from the hamster complement system to evaluate a complement system modulator wherein the complement system modulator is comprised in a group consisting of
(a) activators of C5 or C5a
(b) modulators of C5 activity
(c) modulators of C5a activity, and
(d) modulators of C5aR1 activity.

One embodiment of the invention is a method to use an in vivo complement-system-related-disease hamster animal model to evaluate a complement system modulator.

One embodiment of the invention is a method to use an in vivo complement-system-related-disease hamster animal model to evaluate a complement system modulator, wherein the hamster complement system activation can be reduced by W-54011.

One embodiment of the invention is a method to use an in vivo hamster animal model to evaluate a complement system modulator wherein the hamster model is comprised in a group consisting of:
(a) models for sepsis
(b) models for SIRS
(c) models for organ dysfunction
(d) models for neurodegenerative diseases
(e) models for heart failure
(f) models for renal failure
(g) models for lung failure, and
(h) models for systemic inflammation.

A preferred embodiment of the invention is a method to use an in vivo hamster animal model to evaluate a complement system modulator wherein the hamster model is comprised in a group consisting of:
(a) models for sepsis
(b) models for SIRS
(c) models for organ dysfunction
(d) models for neurodegenerative diseases
(e) models for heart failure
(f) models for renal failure
(g) models for lung failure, and
(h) models for systemic inflammation,
   wherein the hamster complement system activation can be reduced by W-54011.

One embodiment of the invention is an in vivo hamster complement system related disease model to evaluate a complement system modulator in complement system related diseases.

A preferred embodiment of the invention is an in vivo hamster complement system related disease model to evaluate a complement system modulator in a complement system related disease, wherein the hamster complement system activation can be reduced by W-54011.

One embodiment of the invention is an in vivo hamster model to evaluate a complement system modulator, wherein the hamster model is comprised in a group of hamster in vivo models consisting of:
(a) models for sepsis
(b) models for SIRS
(c) models for organ dysfunction
(d) models for neurodegenerative diseases
(e) models for heart failure
(f) models for renal failure
(g) models for lung failure, and
(h) models for systemic inflammation.

One embodiment of the invention is an in vivo hamster model to evaluate a complement system modulator, wherein the hamster model is comprised in a group of hamster in vivo models consisting of:
(a) models for sepsis
(b) models for SIRS
(c) models for organ dysfunction
(d) models for neurodegenerative diseases
(e) models for heart failure
(f) models for renal failure
(g) models for lung failure, and
(h) models for systemic inflammation,
   wherein the hamster complement system activation can be reduced by W-54011. A preferred in vivo hamster model is a hamster CLP sepsis model.

One embodiment of the invention is an in vivo Syrian hamster complement-system-related-disease model to evaluate a complement system modulator in a complement system related disease.

One embodiment of the invention is an in vivo Syrian hamster model to evaluate a complement system modulator wherein the Syrian hamster model is comprised in a group of Syrian hamster in vivo models consisting of:
(a) models for sepsis
(b) models for SIRS
(c) models for organ dysfunction
(d) models for neurodegenerative diseases
(e) models for heart failure
(f) models for renal failure
(g) models for lung failure, and
(h) models for systemic inflammation.

A preferred in vivo Syrian hamster model is a Syrian hamster CLP sepsis model.

One embodiment of the invention is a method to use polypeptides from the hamster complement system, preferably the hamster C5aR1 polypeptide, more preferred the polypeptide of SEQ ID NO: 2, to evaluate a complement system modulator in an in vitro assay wherein the in vitro assay is comprised in a group consisting of:
(a) binding assays
(b) activity assays, and
(c) clinical chemistry parameter assays.

In a preferred object of the invention the complement system modulator is a C5aR1 modulator, even further preferred is a C5aR1 antagonist or inhibitor, preferably the C5aR1 modulator, antagonist or inhibitor is for human medical therapy.

A further preferred embodiment of the invention is an in vivo Syrian hamster CLP sepsis model to evaluate a C5aR1 modulator.

A further preferred embodiment of the invention is a method using an in vivo Syrian hamster CLP sepsis model to evaluate a C5aR1 modulator.

A further preferred embodiment of the invention is an in vivo Syrian hamster CLP sepsis model to evaluate a C5aR1 antagonist.

A further preferred embodiment of the invention is a method using an in vivo Syrian hamster CLP sepsis model to evaluate a C5aR1 antagonist.

A further preferred embodiment of the invention is an in vivo Syrian hamster CLP sepsis model to evaluate a human C5aR1 modulator.

A further preferred embodiment of the invention is a method using an in vivo Syrian hamster CLP sepsis model to evaluate a human C5aR1 modulator.

A further preferred embodiment of the invention is an in vivo Syrian hamster CLP sepsis model to evaluate a human C5aR1 antagonist.

A further preferred embodiment of the invention is a method using an in vivo Syrian hamster CLP sepsis model to evaluate a human C5aR1 antagonist.

Preferably the C5aR1 modulator, antagonist or inhibitor is for human medical therapy.

A further preferred embodiment is the use of the animal models of the invention for the evaluation a human complement modulator, preferably a C5aR1 antagonist.

Another preferred embodiment of the invention is a method of using non-human animal disease model for the evaluation of a complement system modulator for the treatment of a complement-system mediated disease, wherein said animal expresses a polypeptide of the invention which activity can be reduced by W-54011.

A further preferred embodiment of the invention is a method of using non-human animal disease model for the evaluation of a complement system modulator for the treatment of a complement-system mediated disease, wherein said animal expresses a polypeptide of the invention which activity can be reduced by W-54011, wherein the complement-system mediated disease is comprised in a group consisting of sepsis, SIRS, organ dysfunction, neurodegenerative diseases, heart failure, renal failure, lung failure and systemic inflammation.

A further embodiment is a method according to the foregoing embodiments, wherein the non-human animal is a hamster, preferably a Syrian hamster.

A further embodiment is a method according to foregoing embodiments, wherein the non-human disease model is a CLP animal model, preferably a Syrian hamster CLP sepsis model.

A further embodiment is a method according to foregoing embodiments, wherein the complement system modulator is a C5aR1 modulator, preferably a C5aR1 antagonist. Preferably, the C5aR1 modulator or antagonist is for human medical therapy.

A further embodiment is a method according to foregoing embodiments, wherein the disease modulation is monitored by a biomarker, preferably by the measurement of expression levels of IL10, IL6 or IL1b.

The person skilled in the art knows how to use the animal models of the invention for the evaluation of complement modulators, preferably C5aR1 antagonists. If the complement modulator, preferably a C5aR1 antagonist, ameliorates the disease symptom of the animal model (which is observable without treatment with the modulator) the modulator is considered a valuable drug candidate.
1. A C5aR1 polynucleotide, selected from a group consisting of
   (i) nucleic acid molecules encoding a polypeptide comprising the amino acid sequence of SEQ ID NO: 2,
   (ii) nucleic acid molecules comprising the sequence of SEQ ID NO: 1,
   (iii) nucleic acid molecules having the sequence of SEQ ID NO: 1,
   (iv) nucleic acid molecules the complementary strand of which hybridizes under stringent conditions to a nucleic acid molecule of (i), (ii), or (iii); and
   (v) nucleic acid molecules the sequence of which differs from the sequence of a nucleic acid molecule of (iii) due to the degeneracy of the genetic code; wherein the polypeptide encoded by said nucleic acid molecule has C5aR1 activity.
2. A C5aR polypeptide selected from a group consisting of
   (i) polypeptides having the sequence of SEQ ID NO: 2,
   (ii) polypeptides comprising the sequence of SEQ ID NO: 2,
   (iii) polypeptides encoded by C5aR1 polynucleotides as disclosed above; and
   (iv) polypeptides which have at least 85%, 90%, 95%, 98% or 99% identity, wherein said polypeptide has C5aR1 activity.
3. A method of screening for therapeutic agents comprising the steps of
   (i) contacting a test compound with a polypeptide of count 2,
   (ii) detect binding of said test compound to said polypeptide.
4. A method of screening for therapeutic agents comprising the steps of
   (i) determining the activity of a polypeptide of count 2 at a certain concentration of a test compound or in the absence of said test compound,
   (ii) determining the activity of said polypeptide at a different concentration of said test compound.
5. A method of screening for therapeutic agents comprising the steps of
   (i) determining the activity of a polypeptide of count 2 at a certain concentration of a test compound,
   (ii) determining the activity of a said polypeptide at the presence of a compound known to be a regulator of a C5aR1 polypeptide.
6. The method of any of counts 3 to 5, wherein the step of contacting is in or at the surface of a cell.
7. The method of any of counts 3 to 5, wherein the cell is in vitro.
8. The method of any of counts 3 to 5, wherein the step of contacting is in a cell-free system.
9. The method of any of counts 3 to 5, wherein the polypeptide is coupled to a detectable label.
10. The method of any of counts 3 to 5, wherein the compound is coupled to a detectable label.
11. The method of any of counts 3 to 5, wherein the test compound displaces a ligand which is first bound to the polypeptide.
12. The method of any of counts 3 to 5, wherein the polypeptide is attached to a solid support.
13. The method of any of counts 3 to 5, wherein the compound is attached to a solid support.
14. A method of screening for therapeutic agents comprising the steps of
   (i) contacting a test compound with a polynucleotide of count 1,
   (ii) detect binding of said test compound to said polynucleotide.
15. A non-human animal disease model for the evaluation of a complement system modulator for the treatment of a complement-system mediated disease, wherein said animal expresses a polypeptide of count 2.
16. A non-human animal disease model according to count 15, wherein the complement-system mediated disease is comprised in a group consisting of sepsis, SIRS, organ dysfunction, neurodegenerative diseases, heart failure, renal failure, lung failure and systemic inflammation.
17. A non-human animal disease model according to count 15 or 16, wherein the animal is a hamster.
18. A non-human animal disease model according to anyone of counts 15 to 17, wherein the animal is a Syrian hamster.
19. A non-human animal disease model according to anyone of counts 15 to 18, wherein the animal-model is a CLP animal model.
20. A non-human animal disease model according to anyone of counts 15 to 19, wherein the complement system modulator is a C5aR1 modulator.
21. A non-human animal disease model according to anyone of counts 15 to 20, wherein the disease modulation is monitored by a biomarker.
22. A non-human animal disease model according to 21, wherein the biomarker is selected from a group consisting of IL10, IL6 and IL1b
23. Use of a non-human animal expressing a polypeptide according to count 2 as disease model for the characterization of a complement system modulator.
24. Use according to count 23, wherein the animal is a Syrian hamster.
25. Use according to count 23 or 24, wherein the complement system modulator is a C5aR1 modulator.
26. Use according to count 23, 24 or 25, wherein the disease model is selected from the group of disease models consisting of sepsis, SIRS, organ dysfunction, neurodegenerative diseases, heart failure, renal failure, lung failure and systemic inflammation.
27. Use according to anyone of counts 23 to 26, wherein the disease model is selected from the group of disease models consisting of hamster CLP model, hamster Monocrotalin model, hamster chronic myocardial infarction model, hamster DOCA-salt hypertensive model, hamster model for chronic kidney failure, hamster model for dilated cardiomyopathy, hamster BIO14.6 model, hamster inflammation model, hamster models for respiratory distress syndrome, hamster model for Lung emphysema and COPD, hamster acute lung injury model, hamster pneumonia and lung injury model, hamster oxidative stress and renal dysfunction model, hamster model for neurological disorders, and hamster model for cardiac dysfunction.
28. Use according to anyone of counts 23 to 27, wherein the disease modulation is monitored by a biomarker.
29. Use according to count 28, wherein the biomarker is selected from the group consisting of IL10, IL6 and IL1b.
30. Use according to anyone of counts 23 to 29, wherein the complement system modulator is a C5aR1 antagonist.
31. Use according to anyone of counts 23 to 30, wherein the animal-model is a CLP animal model.
32. Use according to anyone of counts 23 to 31, wherein in the disease model the complement system activation can be reduced by compound W-54011, preferably the reduction is at least 30%.
33. Use according to count 32, wherein the reduction is measured in a CLP model.
34. A method of using an animal model according to anyone of counts 15 to 22 for the evaluation of a complement modulator.
35. A method according to count 34 or a use according to anyone of counts 23 to 32, wherein the evaluation comprises comparing the effect of a complement modulator with the effect of a placebo in the animal model.
36. A method according to count 35, wherein the evaluation further comprises the selection of a complement modulator as a drug candidate for the respective disease when the complement modulator ameliorates the disease symptom of the disease model compared to placebo.

### EXAMPLES

### Example 1

### Expression Analysis

Hamster tissues were pulverized by grinding with liquid nitrogen. Total RNA was extracted, DNase I digestion was performed to remove residual genomic DNA and the RNA were reverse transcribed using random hexamer primers. Quantitative TaqMan RT-PCR analysis was performed using the Applied Biosystems PRISM 7900 sequence detection system. The thermal protocol was set to 2 min at 50° C., followed by 10 min at 95° C. and by 40 cycles of 15 s at 95° C. and 1 min at 60° C. Results were normalized to L32 controls, and relative expression was calculated according to the following formula: relative expression=2̂(15−(CT(probe)−CT(L32))). The parameter CT is defined as the cycle number at which the amplification plot passes a fixed threshold above baseline.

### Example 2

### CLP Animal Model

Sepsis and multi organ failure are the most important cause of death among hospitalized patients, with mortality rates ranging from 30 to 70%. Despite advances in supportive care, each year 750,000 people develop sepsis and 225,000 die in the United States alone, and the incidence of sepsis is rising at rates between 1.5% and 8% per year. Sepsis is the result of an acute and systemic immune response to a variety of noxious insults, in particular to bacterial infection. This response leads to the activation of a number of host mediator systems, including the cytokine, leukocyte, and hemostatic networks, each of which may contribute to the pathological sequelae of sepsis. Bacterial sepsis can be induced by cecal ligation puncture (CLP) which induces multiorgan failure [25]. CLP offers a stable model for sepsis mimicking the human situation where colon perforation results in peritonitis which is a common cause for sepsis. CLP sepsis models are described in mice and rats but so far not in gerbils. Therefore, we performed pilot studies to establish the CLP sepsis model in gerbils.

Peritonitis was surgically induced under Isoflurane anesthesia in Syrian Hamster (100-180 g). Midline incision was made in the Linea Alba of the peritoneal cavity and the cecum was exposed. 50% of the cecum was tied off by placing a tight ligature around the cecum. For the CLP model two puncture wounds were made with an 18-gauge needle into the cecum and small amounts of cecal contents were expressed through the wounds, situs was flushed with 0.5 mL sterile saline. Finally, the cecum was replaced into the peritoneal cavity and the laparotomy site was closed. The sham group underwent abdominal surgery; the cecum was exposed and replaced without ligation or puncture of the cecum. Situs was flushed with 0.5 mL sterile saline and the laparotomy site was closed.

Study medication was the C5aR Antagonist W-54011 (CAS number: 405098-33-1) from Cal Biochem Cat #234415. C5aR Antagonist was dissolved in DMSO and diluted with sterile saline solution. The final solution contained 5% DMSO. Two dose groups were tested: 5 mg/kg and 15 mg/kg C5aR Antagonist (C5aR-A). The study medication was given s.c. 30 min before and 2 h after CLP surgery.

Blood samples were obtained under Isoflurane anesthesia from the cavernous sinus with a capillary at different time points to allow measurement of clinical chemistry parameters.

At 24 h after surgery the surviving animals from all groups were anesthetized and exsanguinated by cannulation of the carotid artery. Liver, kidneys, lung, heart and spleen were collected; shock frozen and stored at −80° C. and one specimen of each organ was fixed in formaldehyde. The tissue samples were used for expression analysis of biomarker and IHC studies.

### Example 3

### Use of IL10, IL6 or IL1b as Examples for Biomarkers

Total RNA was isolated from hamster tissues with the Trizol-Reagent protocol according to the manufacturer's specifications (Invitrogen; USA). Total RNA prepared by the Trizol-reagent protocol was treated with DNAse I to remove genomic DNA contamination. For relative quantitation of the mRNA distribution of IL10, IL6 and IL1b, total RNA from each sample was first reverse transcribed. 1 μg of total RNA was reverse transcribed using ImProm-II Reverse Transcription System (Promega, USA) according to the manufactures protocol. The final volume was adjusted to 200 μl with water. For relative quantitation of the distribution of IL10, IL6 or IL1b mRNA the Applied Biosystems PRISM 7900 sequence detection system was used according to the manufacturer's specifications and protocols. PCR reactions were set up to quantitate IL 10, IL6 or IL1b and the housekeeping gene L32. Forward and reverse primers and probes for were designed using the Applied Bioscience ABI Primer Express™ 2.0 software and were synthesized by Eurogentec (Belgium). The forward primer sequence was: Primer1 (IL10: SEQ ID NO: 6; IL6: SEQ ID NO: 15; IL1b: SEQ ID NO: 12; L32: SEQ ID NO: 9). The reverse primer sequence was Primer2 (IL10: SEQ ID NO: 8; IL6: SEQ ID NO: 17; IL1b: SEQ ID NO: 14; L32: SEQ ID NO: 11). Probe1 (IL10: SEQ ID NO: 7; IL6: SEQ ID NO: 16; IL1b: SEQ ID NO: 13; L32: SEQ ID NO: 10), labelled with FAM (carboxyfluorescein succinimidyl ester) as the reporter dye and TAMRA (carboxytetramethylrhodamine) as the quencher, is used as a probe for IL10, IL6, IL1b and L32. The following reagents were prepared in a total of 20 μl: 1×qPCR-MasterMix (Eurogentec; Belgium) and Probe1, IL10 or IL6 or IL1b forward and reverse primers respectively each at 200 nM, 200 nM FAM/TAMRA-labelled probe, and 5 μl of template cDNA. Thermal cycling parameters were 2 min at 50° C., followed by 10 min at 95° C., followed by 40 cycles of melting at 95° C. for 15 sec and annealing/extending at 60° C. for 1 min. Calculation of relative expression: The CT (threshold cycle) value is calculated as described in the “Quantitative determination of nucleic acids” section. deltaCT=CT(IL10 or IL6 or IL1b)−CT132 relative expression=2̂(15−deltaCT). The results of the mRNA-quantification (expression profiling) are shown in FIGS. 6 to 11.

### Example 4

### Hemogram

Blood samples were obtained under light Isoflurane anesthesia from the cavernous sinus with a capillary at different time points/final exsanguination by cannulation of the carotid artery after 24 hrs to allow measurements of differential blood counts. Blood samples for basal blood cell counts were collected from the cavernous sinus one week before study begin. Blood was collected into EDTA tubes and blood cell counts were performed on an automated cell counter.

### Example 5

### Binding Assay

In a receptor binding assay a sample which can be a chemical compound acting as an agonist or antagonist or an antibody acting as an antagonist, is reacted in a reaction mixture simultaneously or in succession with a receptor membrane preparation. A part of the reaction mix is also a compound or peptide labelled radiochemically either with a tritium or 125-iodine label known to bind specifically to the transporter.

First, the receptor membrane preparation is mixed in an appropriate buffer with compounds or antibodies at varying concentrations for which the IC50 value is going to be determined. The receptor/compound or antibody complex is incubated for a specific time until a steady state of binding and dissociation has formed. Then, the radiolabeled compound or peptide is added to the reaction mix. The radiolabeled compound and the non-radio labeled compounds/antibodies compete for the binding site of the receptor.

After reaching the steady state, the unbound radiolabeled compound/peptide is separated from the receptor bound radiolabeled compound/peptide by means of filtration and subsequent washing with an appropriate buffer. The receptor membrane/radiolabeled compound complex is bound to the filtration membrane, which is dried and an appropriate scintillator is added so the radioactive signal can be recorded by a suitable counter.

Alternatively the bound and unbound separation is achieved by binding of the receptor membrane/compound complex to specific beads in a scintillation proximity assay (SPA). Only by binding of the receptor bound radiolabeled compound in a close proximity to the scintillation beads a scintillation signal can be recorded by a suitable counter. Radiolabeled compounds not in such a close proximity as the receptor membrane/compound complex don't give a signal.

The receptor membrane could be prepared from C5aR1 overexpressing cell line. The membrane preparation from cell lines is a state of the art technique and described in the literature. The development of a C5aR1 overexpressing cell line is described in example 6.

### Activity Assay

C5aR1 activity can be determined by a multitude of assays known to the skilled artisan, e.g. by recombinant expression of the C5aR1 and subsequent detection of a known downstream second messenger (29).

### Example 6

### Cloning of Hamster C5aR1 Polynucleotide

To clone the hamster C5a receptor 1 we have done genomic analysis and sequenced the respective fragment according to our findings. C5aR1 poly nucleotides from different glires species (i.e. gerbil (Meriones unguiculatus, AY220495); mouse (Mus musculus, AY220494); rat (Rattus norvegicus, X65862); rabbit (Oryctolagus cuniculus, AAGWO2072785); guinea pig (Cavia porcellus, U86103); pika (Ochotona princeps, AAYZ01433849); squirrel (Spermophilus tridecemlineatus, AAQQ01534263) were aligned. Regions of high homology between these C5aR1 polynucleotides were used to design degenerated oligonucleotides. Using these oligonucleotides genomic hamster DNA was cloned and sequenced. Full length hamster C5aR1 sequence information was obtained by chromosome walking. Full length hamster cDNA was cloned after amplifying hamster lung cDNA by PCR. Mery and Boulay have shown that the amino-terminus of the human C5aR1 polypeptide is important for C5a binding [42]. They showed that replacement of the first 13 residues of C5aR by the corresponding region of FPR resulted in a chimera that was readily transported to the plasma membrane but showed no capability to bind C5a. FPR and C5aR1 have an overall sequence identity of 34%.

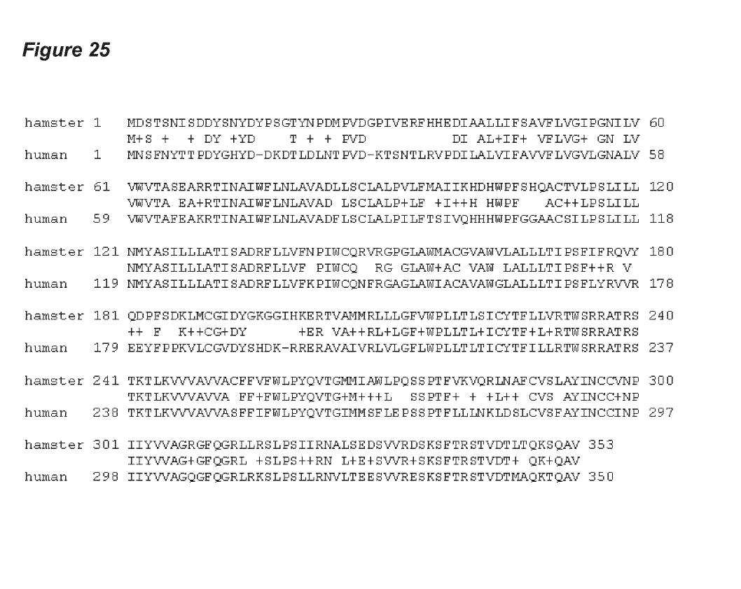

However, sequence comparison of hamster and human C5aR1 polypeptide sequence reveals low sequence identify and homology especially at the amino-terminus of the receptors (see FIG. 25), especially the first 13 amino acids of human and hamster C5aR1 has a sequence identity of only 30%. It is therefore surprising, that despite sequence differences in the amino-terminal region of hamster and human C5aR1 polypeptide hamster C5aR1 is a functional C5a receptor and is activated by human C5a ligand. Moreover, receptor activation of hamster C5aR1 can be reduced by human C5aR1 receptor antagonist W-54011. Surprisingly, we could identify the hamster C5a receptor as human-like.

### Development of a Recombinant Cell Line or Host Expressing Hamster C5aR1

Expression of hamster C5aR1 is accomplished by subcloning the cDNAs into appropriate expression vectors and transfecting the vectors into expression hosts such as, e.g., E. coli. In a particular case, the vector is engineered such that it contains a promoter for β-galactosidase, upstream of the cloning site, followed by sequence containing the amino-terminal Methionine and the subsequent seven residues of β-galactosidase. Immediately following these eight residues is an engineered bacteriophage promoter useful for artificial priming and transcription and for providing a number of unique endonuclease restriction sites for cloning.

Induction of the isolated, transfected bacterial strain IPTG using standard methods produces a fusion protein corresponding to the first seven residues of β-galactosidase, about 15 residues of “linker”, and the peptide encoded within the cDNA. Since cDNA clone inserts are generated by an essentially random process, there is probability of 33% that the included cDNA will lie in the correct reading frame for proper translation. If the cDNA is not in the proper reading frame, it is obtained by deletion or insertion of the appropriate number of bases using well known methods including in vitro mutagenesis, digestion with exonuclease III or mung bean nuclease, or the inclusion of an oligonucleotide linker of appropriate length.

The C5aR1 cDNA is shuttled into other vectors known to be useful for expression of proteins in specific hosts. Oligonucleotide primers containing cloning sites as well as a segment of DNA (about 25 bases) sufficient to hybridize to stretches at both ends of the target cDNA is synthesized chemically by standard methods. These primers are then used to amplify the desired gene segment by PCR. The resulting gene segment is digested with appropriate restriction enzymes under standard conditions and isolated by gel electrophoresis. Alternately, similar gene segments are produced by digestion of the cDNA with appropriate restriction enzymes. Using appropriate primers, segments of coding sequence from more than one gene are ligated together and cloned in appropriate vectors. It is possible to optimize expression by construction of such chimeric sequences.

Suitable expression hosts for such chimeric molecules include, but are not limited to, mammalian cells such as Chinese Hamster Ovary (CHO) and human 293 cells, insect cells such as Sf9 cells, yeast cells such as Saccharomyces cerevisiae and bacterial cells such as E. coli. For each of these cell systems, a useful expression vector also includes an origin of replication to allow propagation in bacteria, and a selectable marker such as the β-lactamase antibiotic resistance gene to allow plasmid selection in bacteria. In addition, the vector may include a second selectable marker such as the neomycin phosphotransferase gene to allow selection in transfected eukaryotic host cells. Vectors for use in eukaryotic expression hosts require RNA processing elements such as 3′ polyadenylation sequences if such are not part of the cDNA of interest.

Additionally, the vector contains promoters or enhancers which increase gene expression. Such promoters are host specific and include MMTV, SV40, and metallothionine promoters for CHO cells; trp, lac, tac and T7 promoters for bacterial hosts; and alpha factor, alcohol oxidase and PGH promoters for yeast. Transcription enhancers, such as the rous sarcoma virus enhancer, are used in mammalian host cells. Once homogeneous cultures of recombinant cells are obtained through standard culture methods, large quantities of re [symbol] combinantly produced C5aR1 are recovered from the conditioned medium and analyzed using chromatographic methods known in the art. For example, C5aR1 can be cloned into the expression vector pcDNA3, as exemplified herein. This product can be used to transform, for example, HEK293 or COS by methodology standard in the art. Specifically, for example, using Lipofectamine (Gibco BRL catalog no. 18324-020) mediated gene transfer.

### Hamster C5aR1 is a Human C5a Receptor:

CHO K1 cell lines expressing mitochondrial Clytin (CHOmtCly) were co-transfected with the expression vector pcDNA3 harbouring the cDNA of hamster C5aR1 und pcDNA3-Galpha16 to allow the measurement of C5aR1 signalling via Calcium release (generated cells are named CHOmtCly_hamster-C5aR1/Galpha16). Control cells (CHOmtCly_pcDNA3) were generated by transfection of CHOmtCly cells with a corresponding amount of empty pcDNA3 vector only. In brief, 10⁶ cells were transfected with 2 μg DNA by use of the Nucleofector (Amaxxa) program U-27. Transfected cells were seeded in 384-well MTP with a density of 250 cells per well. After 24 h incubation at 37° C. selection media containing a final concentration of 1 mg/ml G418 was added. After one week of selection plates were duplicated and tested for C5aR1 signalling. Cells were loaded with Coelenterazine for 3 h, than buffer or the C-terminal peptide of human C5a (Bachem H3462) was added at a final concentration of 7 μM. Luminescence (RLU) was measured for 60 seconds.

For activity measurement, three CHOmtCly_hamster-C5aR1/Galpha16 cell lines and three CHOmtCly cDNA3 cell lines were analyzed, respectively.

### Results:

Stimulation of the hamster C5aR1 expressing cell lines CHOmtCly_hamster-C5aR1/Galpha16 with buffer only resulted in RLU values of 127, 206 and 146, respectively. Wherein, stimulation with Bachem peptide 83462 resulted in (RLU) values of 247943, 243143, and 203151, respectively. Whereas, incubation of the control cells CHOmtCly_pcDNA3 with peptide H3462 resulted in much lower RLU values (for buffer: 589, 268, and 200; for H3462: 6686, 2857, and 1976). This clearly demonstrates that the hamster C5aR1 protein of the invention is a functional C5aR1 receptor and is stimulatable by human C5a peptide.

### Example 7

### Identification of Further Suitable Animal Models

The polynucleotides or polypeptides of the invention can be used to identify further complement system suitable animal models. Therefore, animal tissue is pulverized by grinding with liquid nitrogen. Chromosomal DNA is extracted, digested with a restriction endonuclease, and size separated by gel electrophoresis. The gel is blotted on a membrane and probed with a labelled polynucleotide of the invention. The labelled probe detects the presence of a C5aR1 receptor polynucleotide of the invention in a further animal. The so characterized animal expresses a C5aR1 polypeptide of the invention, hence a further C5aR1 polypeptide inhibitable by W-54011. A further animal is provided suitable for the characterization of a complement system modulator.

### Example 8

### Further Animal Models to Test Complement Modulators

### Hamster Model for Pulmonary Arterial Hypertension and Heart Failure

Adult hamsters are treated by single subcutaneous injection of either 60 mg/kg Monocrotaline or vehicle. To test C5aR1 or complement modulators animals (or groups) are treated additionally with compounds (as W54011 as positive control). The Monocrotaline (MCT)-treated animal model is a widely used model for pulmonary arterial hypertension and heart failure. After subcutaneous injection the pyrrolizidine alkaloid MCT is activated by the liver to the toxic MCT pyrrole, which causes endothelial injury in the pulmonary vasculature within few days with subsequent remodeling of small pulmonary arteries (de novo muscularization and medial hypertrophy). In the present study, MCT induces severe, progressive pulmonary hypertension in all animals (treated with MCT only). Four weeks after a single MCT injection, the animals display elevated right ventricular systolic pressure accompanied by a reduction of systemic arterial pressure, cardiac index, arterial oxygenation and central venous oxygen saturation. A candidate C5aR1 or complement inhibitor treated diseased animal does show reduced right ventricular systolic pressure accompanied by an increased systemic arterial pressure, cardiac index, arterial oxygenation and central venous oxygen saturation compared to non-treated diseased animals.

### Hamster Chronic Myocardial Infarction Model

In the chronic myocardial infarction model left coronary artery ligation is performed under isoflurane anaesthesia. Following a left thoractomy at the fourth intercostal space, the pericardium is opened and the heart briefly exteriorized. The left coronary artery (LAD) is chronically ligated. In sham operated animals the LAD stays open. The chest is closed and animals are weaned from the ventilator and placed in cages with free access to food and water. One week after LAD occlusion application of test compounds (C5aR1 or complement modulators) is started. Heart tissue and plasma samples are analyzed 9 weeks after induction of the infarct towards plasma markers, infarct size and expression profiles. A candidate C5aR1 or complement inhibitor treated diseased animal does show reduced infarct size compared to non-treated diseased animals

### Hamster DOCA-Salt Hypertensive Animal Model for Left Ventricular Hypertrophy

The DOCA-salt hypertensive animal model is a well-established model of left ventricular hypertrophy. Uninephrectomized animals are given 1% NaCl in drinking water and subcutaneous injections of deoxycorticosterone acetate (for example: DOCA, 30 mg/kg once weekly) for four weeks. To test C5aR1 or complement modulators animals (or groups) are treated additionally with compounds (as W54011 as positive control). Untreated (without DOCA) animals without uninephrectomy serve as control animals. After four weeks DOCA-salt hamsters show a significant increase in the tibia length-corrected left ventricular mass. A candidate C5aR1 or complement inhibitor treated diseased animal does show reduced tibia length-corrected left ventricular mass compared to non-treated diseased animals.

### Hamster Model for Chronic Kidney Failure

The 5/6 nephrectomy is performed in adult hamsters by a nephrectomy of the right kidney and resection of two thirds of the left kidney. Animals are treated with C5aR1 or complement modulators. Serum creatine is measured using a Creatinine Reagent Assay (Raichem, San Marcos, Calif., USA) according to the manufacturer protocol. Hematuria and proteinuria are measured using DiaScreen (Chronimed Inc., Minnetonka, Minn., USA) reagent strips in the urine. For kidney morphology, hematoxylin and eosin-stained 3-m sections of paraffin-embedded kidneys are analyzed. In each control animal, the entire area of longitudinal sections of one kidney is evaluated. A candidate C5aR1 or complement inhibitor treated diseased animal does show reduced hematuria, proteinuria, creatine levels and normalized kidney morphology compared to non-treated diseased animals. Those effects could be appear in combination or single.

### Hamster Model for Dilated Cardiomyopathy

Hamster animal models for the development and progression of dilated cardiomyopathy in the Syrian Cardiomyopathic Hamster (SCH) model are described in the literature [30] and could be used for the testing of C5aR1 inhibitors or complement modulators.

### BIO14.6 Hamster Model

Hamster animal models for the development and progression of autosomal recessive cardiomyopathy and progressive myocardial necrosis and heart failure and arrhythmia are described in the literature [31, 36] and could be used for the testing of C5aR1 inhibitors or complement modulators.

### Hamster Inflammation Model

Hamster models for the characterization of inflammation, immune response and infections are described in the literature [32] and could be used for the testing of C5aR1 inhibitors and complement modulators.

### Hamster Models for Respiratory Distress Syndrome (ARDS)

Hamster models for the characterization of respiratory distress syndrome are described in the literature [33] and could be used for the testing of C5aR1 inhibitors and complement modulators.

### Hamster Model for Lung Emphysema and COPD

Hamster models for the characterization of lung emphysema and COPD are described in the literature [34] and could be used for the testing of C5aR1 inhibitors and complement modulators.

### Hamster Acute Lung Injury Model

Hamster models for the characterization of acute lung injury are described in the literature [35, 38] and could be used for the testing of C5aR1 inhibitors and complement modulators.

### Hamster Pneumonia and Lung Injury Model

Hamster models for the characterization of pneumonia and lung injury are described in the literature [37] and could be used for the testing of C5aR1 inhibitors and complement modulators.

### Hamster Oxidative Stress and Renal Dysfunction Model

Hamster models for the characterization of oxidative stress and renal dysfunction are described in the literature [39] and could be used for the testing of C5aR1 inhibitors and complement modulators.

### Hamster Model for Neurological Disorders

Hamster models for the characterization of neurological disorders are described in the literature [40] and could be used for the testing of C5aR1 inhibitors and complement modulators.

### Hamster Model for Cardiac Dysfunction

Hamster models for the characterization of cardiac dysfunction are described in the literature [41] and could be used for the testing of C5aR1 inhibitors and complement modulators.

### REFERENCES

1. Janeway C A Jr., Travers P, Walport M, Shlomchik M J (2001). Immunobiology. (5th ed.). Garland Publishing
2. Goldman A S, Prabhakar B S (1996). The Complement System. in: Baron's Medical Microbiology (Baron S et al., eds.) (4th ed.). Univ of Texas Medical Branch.
3. Monk P N, Scola A-M, Madala P, Fairlie D P (2007). “Function, structure and therapeutic potential of complement C5a receptors”. British Journal of Pharmacology 152: 429-48
4. Svetlana Bureeva, Julian Andia-Pravdivy and Alexander Kaplun; Drug design using the example of the complement system inhibitors' development; DDT; 10, 22; November 2005
5. Peter A. Ward (2009), J Mol Med 87:375-378, Functions of C5a receptors
6. Gerard N P, Gerard C (1991) The chemotactic receptor for human C5a anaphylatoxin. Nature 349:614-617
7. Boulay F, Tardif M, Brouchon L, Vignais P (1990) Synthesis and use of a novel N-formyl peptide derivative to isolate a human Nformyl peptide receptor cDNA. Biochem Biophys Res Commun 168:1103-1109
8. Strainic M G, Liu J, Huang D, An F, Lalli P N, Muqim N, Shapiro V S, Dubyak G R, Heeger P S, Medof M E (2008) Locally produced complement fragments C5a and C3a provide both costimulatory and survival signals to naïve CD4+ T cells. Immunity 28:425-435
9. Hawlisch H, Wills-Karp M, Karp C L, Köhl J (2004) The anaphylatoxins bridge innate and adaptive immune responses in allergic asthma. Mol Immunol 41:123-131
10. Lee H, Whitfeld P L, Mackay C R (2008) Receptors for complement C5a. The importance of C5aR and the enigmatic role of C5L2. Immunol Cell Biol 86:153-160
11. Okinaga S, Slattery D, Humbles A, Zsengeller Z, Morteau O, Kinrade M B, Brodbeck R M, Krause J E, Choe H R, Gerard N P, Gerard C (2003) C5L2, a nonsignaling C5a binding protein. Biochemistry 42:9406-9415
12. Maria I. Fonseca, Rahasson R. Ager, Shu-Hui Chu, Ozkan Yazan, Sam D. Sanderson, Frank M. LaFerla, Stephen M. Taylor, Trent M. Woodruff, and Andrea J. Tenner, Journal of Immunology, 2009, 183: 1375-1383, Treatment with a C5aR Antagonist Decreases Pathology and Enhances Behavioral Performance in Murine Models of Alzheimer's Disease
13. Andreas Klosa,l, Andrea J. Tennerb,l, Kay-Ole Johswicha, Rahasson R. Agerb, Edimara S. Reis c, Jörg Köhl Molecular Immunology 46 (2009) 2753-2766, The role of the anaphylatoxins in health and disease
14. Kay Johswich, Myriam Martin, Andre Bleich, Michael Kracht, Oliver Dittrich-Breiholz, J. Engelbert Gessner, Sebastian Suerbaum, Elisabeth Wende, Claudia Rheinheimer, Andreas Klos, Inflamm Bowel Dis 2009, Role of the C5a Receptor (C5aR) in Acute and Chronic Dextran Sulfate-Induced Models of Inflammatory Bowel Disease
15. A. G. Lewis, G. Köhl, Q. Ma, P. Devarajan, J. Köhl, Clinical and Experimental immunology, 153: 117-126 (2008), Pharmacological targeting of C5a receptors during organ preservation improves kidney graft survival
16. Daniel Rittirsch, Michael A Flierl, Brian A Nadeau, Danielle E Day, Markus Huber-Lang, Charles R Mackay, Firas S Zetoune, Norma P Gerard, Katherine Cianflone, Joerg Koehl, Craig Gerard, J Vidya Sarmal, Peter Ward, Nature medicine 14(5) 2008, Functional roles for C5a receptors in sepsis
17. Konstantinos Kambas, Maciej M. Markiewski, Ioannis A. Pneumatikos, Stavros S. Rafail, Vassiliki Theodorou, Dimitrios Konstantonis, Ioannis Kourtzelis, Michael N. Doumas, Paola Magotti, Robert A. DeAngelis, John D. Lambris, Konstantinos D. Ritis, The Journal of Immunology, 2008, 180: 7368-7375, C5a and TNF-a Up-Regulate the Expression of Tissue Factor in Intra-Alveolar Neutrophils of Patients with the Acute Respiratory Distress Syndrome
18. Prathit A Kulkarni, Vahid Afshar-Kharghan, Biologics: Targets & Therapy 2008:2(4) 671-685, Anticomplement therapy
19. Laszlo M. Hoesel a, Andreas D. Niederbichler b, Peter A. Ward, Molecular Immunology 44 (2007) 95-102, Complement-related molecular events in sepsis leading to heart failure
20. Denis W. Harkin, Alex Romaschin, Stephen M. Taylor, Barry B. Rubin, Thomas F. Lindsay, J Vasc Surg 2004; 39:196-206, Complement C5a receptor antagonist attenuates multiple organ injury in a model of ruptured abdominal aortic aneurysm
21. Robert W. Schrier, Wei Wang, N Engl J Med 2004; 351:159-69, Acute Renal Failure and Sepsis
22. Allan K Wong, Stephen M Taylor, David P Fairlie, IDrugs 1999 Vol 2 No 7, Development of C5a receptor antagonists
23. Stephen M. Waters‡, Robbin M. Brodbeck‡, Jeremy Steflik‡, Jianying Yu‡, Carolyn Baltazar, Amy E. Peck, Daniel Severance, Lu Yan Zhang, Kevin Currie, Bertrand L. Chenard, Alan J. Hutchison, George Maynard, James E. Krause, J Biol Chem. 2005 Dec. 9; 280(49):40617-23, Molecular Characterization of the Gerbil C5a Receptor and Identification of a Transmembrane Domain V Amino Acid That Is Crucial for Small Molecule Antagonist Interaction
24. Patrick Nef, DDT Vol. 6, No. 15 (Suppl.), 2001, Key animal models for the identification and validation of drug targets
25. Corral J, Yelamos J, et al. Role of Lipopolysaccharide and Cecal Ligation and Puncture on Blood Coagulation and Inflammation in Sensitive and Resistant Mice Models 2005 April; 166(4):1089-98
26. American College of Chest Physicians/Society of Critical Care Medicine Consensus Conference: definitions for sepsis and organ failure and guidelines for the use of innovative therapies in sepsis”. Crit. Care Med. 20 (6): 864-74.1992
27. “Septic shock; current pathogenetic concepts from a clinical perspective”. Medical Science Monitor: International Medical Journal of Experimental and Clinical Research 11 (3): RA76-85.
28. Rippe, James M.; Irwin, Richard S.; Cerra, Frank B (1999). Irwin and Rippe's intensive care medicine. Philadelphia: Lippincott-Raven
29. Higginbottom A et al, J Biol Chem. 2005 May 6; 280(18):17831-40, Comparative agonist/antagonist responses in mutant human C5a receptors define the ligand binding site.
30. Crespo M J, Cangiano J L, Altieri P I, Escobales N.; Aliskiren Improves Left Ventricular Dysfunction and Reduces Cardiac Dilation in Syrian Cardiomyopathic Hamsters.; J Cardiovasc Pharmacol. 2012 Feb. 24
31. Nigro V, Okazaki Y, Belsito A, Piluso G, Matsuda Y, Politano L, Nigro G, Ventura C, Abbondanza C, Molinari A M, Acampora D, Nishimura M, Hayashizaki Y, Puca G A.; Identification of the Syrian hamster cardiomyopathy gene.; Hum Mol Genet. 1997 April; 6(4):601-7.
32. Zivcec M, Safronetz D, Haddock E, Feldmann H, Ebihara H.; Validation of assays to monitor immune responses in the Syrian golden hamster (Mesocricetus auratus).; I Immunol Methods. 2011 May 31; 368(1-2):24-35
33. Hammerbeck C D, Hooper J W.; T cells are not required for pathogenesis in the Syrian hamster model of hantavirus pulmonary syndrome; J Virol. 2011 October; 85(19):9929-44.
34. van Flees H, Ottenheijm C, Ennen L, Linkels M, Dekhuijzen R, Heunks L.; Proteasome inhibition improves diaphragm function in an animal model for COPD.; Am J Physiol Lung Cell Mol Physiol. 2011 July; 301(1)1110-6
35. Vecchiola A, de la Llera J F, Ramirez R, Olmos P, Herrera C I, Borzone G.; Differences in acute lung response to elastase instillation in two rodent species may determine differences in severity of emphysema development.; Am J Physiol Regul Integr Comp Physiol. 2011 July; 301(1):R148-58.
36. Sakamoto A, Sugamoto Y.; Identification of a novel aldose reductase-like gene upregulated in the failing heart of cardiomyopathic hamster.; Mol Cell Biochem. 2011 July; 353(1-2):275-81.
37. Bhaysar T, Liu M, Liu X, Cantor J.; Aerosolized recombinant human lysozyme enhances the bactericidal effect of tobramycin in a hamster model of Pseudomonas aeruginosa-induced pneumonia.; Exp Lung Res. 2011 November; 37(9):536-41.
38. Takano Y (Mitsuhashi H, Ueno K.; 1α,25-Dihydroxyvitamin D₃ inhibits neutrophil recruitment in hamster model of acute lung injury.; Steroids. 2011 November; 76(12):1305-9. Epub 2011 Jul. 2.
39. Maeda Y, Inoguchi T, Takei R, Sawada F, Sasaki S, Fujii M, Kobayashi K, Urata H, Nishiyama A, Takayanagi R.; Inhibition of chymase protects against diabetes-induced oxidative stress and renal dysfunction in hamsters.; Am J Physiol Renal Physiol. 2010 December; 299(6):F1328-38. Epub 2010 Sep. 29.
40. Groschup M H, Buschmann A.; Rodent models for prion diseases.; Vet Res. 2008 July-August; 39(4):32. Epub 2008 Feb. 15
41. Ichihara S, Yamada Y, Ichihara G, Kanazawa H, Hashimoto K, Kato Y, Matsushita A, Oikawa S, Yokota M, Iwase M.; Attenuation of oxidative stress and cardiac dysfunction by bisoprolol in an animal model of dilated cardiomyopathy.; Biochem Biophys Res Commun. 2006 Nov. 10; 350(1):105-13.
42. Mery L, Boulay F.; The NH2-terminal region of C5aR but not that of FPR is critical for both protein transport and ligand binding.; J Biol Chem. 1994 Feb. 4; 269(5):3457-63.

1. A C5aR1 polynucleotide, selected from a group consisting of
(i) nucleic acid molecules encoding a polypeptide comprising the amino acid sequence of SEQ ID NO: 2,
(ii) nucleic acid molecules comprising the sequence of SEQ ID NO: 1,
(iii) nucleic acid molecules having the sequence of SEQ ID NO: 1,
(iv) nucleic acid molecules the complementary strand of which hybridizes under stringent conditions to a nucleic acid molecule of (i), (ii), or (iii); and
(v) nucleic acid molecules the sequence of which differs from the sequence of a nucleic acid molecule of (iii) due to the degeneracy of the genetic code;
wherein the polypeptide encoded by said nucleic acid molecule has C5aR1 activity.

2. A C5aR polypeptide selected from a group consisting of
(i) polypeptides having the sequence of SEQ ID NO: 2,
(ii) polypeptides comprising the sequence of SEQ ID NO: 2,
(iii) polypeptides encoded by C5aR1 polynucleotides as disclosed above; and
(iv) polypeptides which have at least 85%, 90%, 95%, 98% or 99% identity,
wherein said polypeptide has C5aR1 activity.

3. A method of screening for therapeutic agents comprising the steps of
(i) contacting a test compound with a polypeptide of claim 2,
(ii) detect binding of said test compound to said polypeptide.

4. A method of screening for therapeutic agents comprising the steps of
(i) determining the activity of a polypeptide of claim 2 at a certain concentration of a test compound or in the absence of said test compound,
(ii) determining the activity of said polypeptide at a different concentration of said test compound.

5. A method of screening for therapeutic agents comprising the steps of
(i) determining the activity of a polypeptide of claim 2 at a certain concentration of a test compound,
(ii) determining the activity of a said polypeptide at the presence of a compound known to be a regulator of a C5aR1 polypeptide.

6. The method of claim 3, wherein the step of contacting is in or at the surface of a cell.

7. Use of a non-human animal expressing a polypeptide according to claim 2 as disease model for the characterization of a complement system modulator.

8. Use according to claim 7, wherein the animal is a Syrian hamster.

9. Use according to claim 7, wherein the complement system modulator is a C5aR1 modulator.

10. Use according to claim 7, wherein the disease model is selected from the group of disease models consisting of sepsis, SIRS, organ dysfunction, neurodegenerative diseases, heart failure, renal failure, lung failure and systemic inflammation.

11. Use according to claim 7, wherein the disease model is selected from the group of disease models consisting of hamster CLP model, hamster Monocrotalin model, hamster chronic myocardial infarction model, hamster DOCA-salt hypertensive model, hamster model for chronic kidney failure, hamster model for dilated cardiomyopathy, hamster BIO14.6 model, hamster inflammation model, hamster models for respiratory distress syndrome, hamster model for Lung emphysema and COPD, hamster acute lung injury model, hamster pneumonia and lung injury model, hamster oxidative stress and renal dysfunction model, hamster model for neurological disorders, and hamster model for cardiac dysfunction.

12. Use according to claim 7, wherein the disease modulation is monitored by a biomarker.

13. Use according to claim 12, wherein the biomarker is selected from the group consisting of IL10, IL6 and IL1b.

14. Use according to claim 7, wherein the complement system modulator is a C5aR1 antagonist.

15. Use according to claim 7, wherein the animal-model is a CLP animal model.

16. The method of claim 4, wherein the step of contacting is in or at the surface of a cell.

17. The method of claim 5, wherein the step of contacting is in or at the surface of a cell.


SEQUENCE LISTING (17 sequences)

SEQ ID NO: 1 - DNA, 1062 bp, Mesocricetus auratus
atggattccaccagcaacatctccgatgactacagcaactatgattaccccagtgggacctataaccctgacatgcctgtggatggtcccatcgttgagcggtttcatcatgaagatattgcagccctgctcatcttctcggctgtgttcctggtgggaatccctgggaacatcctggtggtgtgggtgacagcatccgaggccagacgtaccatcaatgccatctggttcctgaatctggcggtggccgacctcctctcctgcttggcactgcctgtcctattcatggccatcataaaacacgaccattggcctttcagccaccaggcctgtacggtcctgccctcactcattctgcttaacatgtatgccagtatcctgctgctggccaccattagcgctgaccgcttcctgctggtattcaaccccatctggtgtcagagggtccgggggcccggcctggcgtggatggcctgtggagtggcctgggtcttagcgctactcctcaccatcccatccttcatattccgtcaggtgtaccaagaccccttctccgataagttgatgtgtggcattgactacgggaagggtggcatccacaaggagaggacggtggccatgatgcgcctgctactgggctttgtgtggcctctgctcactctcagtatctgctacaccttcctcctggtacgaacctggagtcgcagggccacacgctccaccaagacgctcaaggtggtggtggccgtggtggcctgtttcttcgtcttctggctgccttaccaggtgacagggatgatgattgcctggcttccccagtcctcgcccaccttcgtgaaggtgcagaggctgaacgccttctgtgtgtccctggcctacatcaactgctgtgtcaaccctatcatctatgtcgtggctggccggggcttccaagggcggcttctcaggtcactccccagcatcatccgaaacgccctctcagaggactcggtggtcagggacagcaagtctttcactcgctccacggtggacaccttgacccagaagagtcaagcggtgtag

SEQ ID NO: 2 - protein, 353 aa, Mesocricetus auratus
MDSTSNISDDYSNYDYPSGTYNPDMPVDGPIVERFHHEDIAALLIFSAVFLVGIPGNILVVWVTASEARRTINAIWFLNLAVADLLSCLALPVLFMAIIKHDHWPFSHQACTVLPSLILLNMYASILLLATISADRFLLVFNPIWCQRVRGPGLAWMACGVAWVLALLLTIPSFIFRQVYQDPFSDKLMCGIDYGKGGIHKERTVAMMRLLLGFVWPLLTLSICYTFLLVRTWSRRATRSTKTLKVVVAVVACFFVFWLPYQVTGMMIAWLPQSSPTFVKVQRLNAFCVSLAYINCCVNPIIYVVAGRGFQGRLLRSLPSIIRNALSEDSVVRDSKSFTRSTVDTLTQKSQAV

SEQ ID NO: 3 - DNA, 18 bp, Artificial Sequence
  primer
atgatgcgcctgctactg

SEQ ID NO: 4 - DNA, 25 bp, Artificial Sequence
  primer
tttgtgtggcctctgctcactctca

SEQ ID NO: 5 - DNA, 20 bp, Artificial Sequence
  primer
tcgtaccaggaggaaggtgt

SEQ ID NO: 6 - DNA, 18 bp, Artificial Sequence
  primer
atgccccaggcagagaac

SEQ ID NO: 7 - DNA, 26 bp, Artificial Sequence
  primer
tcaaggagcatttgaactccctagga

SEQ ID NO: 8 - DNA, 20 bp, Artificial Sequence
  primer
tcctgagggtcttcagcttc

SEQ ID NO: 9 - DNA, 17 bp, Artificial Sequence
  primer
ctggcggaaacccagag

SEQ ID NO: 10 - DNA, 25 bp, Artificial Sequence
  primer
agattcaagggccagatcctgatgc

SEQ ID NO: 11 - DNA, 21 bp, Artificial Sequence
  primer
tgcttggttttcttgttgctc

SEQ ID NO: 12 - DNA, 20 bp, Artificial Sequence
  primer
cagggagaaacaagcaacaa

SEQ ID NO: 13 - DNA, 25 bp, Artificial Sequence
  primer
ccctgggcctaaagggaaagaacct

SEQ ID NO: 14 - DNA, 21 bp, Artificial Sequence
  primer
cacctttcatgacacaggaca

SEQ ID NO: 15 - DNA, 21 bp, Artificial Sequence
  primer
tggagtttgtgacgaacaatg

SEQ ID NO: 16 - DNA, 27 bp, Artificial Sequence
  primer
aagacaaagccagagtcattcagagca

SEQ ID NO: 17 - DNA, 23 bp, Artificial Sequence
  primer
gatctgacttagggttttgatgg